(19) United States
(12) Patent Application Publication
(10) Pub. No.: US 2024/0325067 A1
(43) Pub. Date: Oct. 3, 2024

(54) METHOD AND DEVICE FOR TREATING THE SKIN

(71) Applicant: Venus Concept Inc., Toronto (CA)

(72) Inventors: Yoni Iger, Haifa (IL); Ognjen Petrovic, San Jose, CA (US); Haim Epshtein, Hagvora (IL); Cliff Oostman, Hansville, WA (US); Vadim Polyakov, Petach Tikva (IL)

(73) Assignee: Venus Concept Inc., Toronto, ON (CA)

(21) Appl. No.: 18/550,901

(22) PCT Filed: Mar. 15, 2022
(86) PCT No.: PCT/US2022/020338

§ 371 Date: Sep. 15, 2023

Related U.S. Application Data
(60) Provisional application No. 63/161,471, filed on Mar. 16, 2021.

(51) Int. Cl. A61B 18/04; A61B 18/00; A61B 18/14; A61B 18/18; A61B 34/30; A61B 90/00; G16H 20/40

(52) U.S. Cl. CPC A61B 18/04; A61B 18/18; A61B 34/30; A61B 90/03; A61B 90/06; A61B 90/37; G16H 20/40; A61B 2018/00005; A61B 2018/00202; A61B 2018/00321; A61B 2018/00452; A61B 2018/00583; A61B 2018/00589; A61B 2018/00702; A61B 2018/00738; A61B 2018/00791; A61B 2018/00875; A61B 2018/00994; A61B 2018/1425; A61B 2090/033; A61B 2090/064; A61B 2090/3735; A61B 2090/378; A61B 2218/007

(57) ABSTRACT

A method of directional skin tightening by fractional treatment of the skin is provided. The method includes producing a plurality of excised tissue portions in a region of skin tissue. The method further includes applying contraction or expansion tension to the region of skin tissue in at least one predetermined direction. Applying the contraction or expansion tension to the region of skin tissue promotes collagen growth and provides directional skin tightening in the region of skin tissue.

### CROSS-REFERENCE TO RELATED APPLICATION

The present application claims the benefit of priority to U.S. Provisional No. 63/161,471 dated Mar. 16, 2021, the entire contents of which are incorporated herein by reference.

### BACKGROUND OF THE INVENTION

This invention relates to methods and devices for skin treatment. More, specifically, this invention relates to methods and devices for skin coring and tightening that would benefit from endorsing collagen growth and providing directional skin tightening in said skin tissue thus, providing skin restoration or tightening.

Excess tissue and skin laxity are of wide concern in aesthetic medicine. Many patients currently undergo invasive surgical treatments such as face lift to treat such conditions.

### SUMMARY OF THE INVENTION

The present invention discloses minimally invasive device for skin tightening. Furthermore, directional tightening device will excise microcores of skin to provide the desired aesthetic look.

In aesthetic medicine, elimination of excess tissue and/or skin laxity is an important concern that affects more than 25% of the U.S. population. Conventional surgical therapies (e.g., a face lift, brow lift, or breast lift) can be effective but are often invasive, inconvenient, and expensive, while scarring limits their applicability.

Removing 5%-15% of skin in an area through excising a multitude of <1 mm diameter cores of dermis and applying directional compression elastic bandages has been shown to provide skin tightening that can be tuned in a desired direction without (noticeable) scarring. An automated robotic dermal micro-coring system with machine vision and robotic precision can deliver accuracy, repeatability, and efficiency that provides high value to medical clinics.

Methods using energy sources (e.g., laser, non-coherent light, radiofrequency, or ultrasound) can be effective at improving the architecture and the texture of the skin but are much less effective at tightening the skin or reducing skin laxity. Neurotoxins, such as botulinum toxin, reduce the formation of dynamic wrinkles by paralysis of the injected muscles, but such toxins have minimal or no effect on skin tightness or laxity. Finally, dermal fillers, such as hyaluronic acid, are injected in the dermal layer to smooth out wrinkles and improve contours, but such fillers do not tighten or reduce laxity of the skin. Thus, surgical therapies remain the gold standard for lifting and/or tightening skin.

Rotational fractional resection (“RFR”) is a procedure which may be used to achieve focal aesthetic contouring by removing lax skin and excess fat tissue from a patient. Skin may be removed by the use of a rotating micro-coring punch, which is a hollow, sharpened tube which excises full thickness dermal resections. Such punch has been adapted to treat, among other conditions, scars, acne scars, lines, wrinkles, stretch marks, melasma, and to improve skin texture and tighten the skin. As the punch create tiny punctures in the top layer of the skin; such puncture triggers the body's healing process; thereby such devices give the treated area a chance to heal with less discoloration and/or deformation and greater smoothness on the surface.

However, such methods are not problem-free and there is still a need to enhance efficacy thereof. Thus, there is a need for improved methods and devices that increase the effectiveness of such minimally-invasive techniques. Furthermore, there is still a long felt need for an automated robotic system for dermal micro-coring to be used in minimally invasive directional skin tightening procedures.

This invention relates to methods and devices for skin treatment. More specifically, this invention relates to methods and devices for skin coring and tightening that would benefit from endorsing collagen growth and providing directional skin tightening in said skin tissue thus, providing skin restoration or tightening.

It is one object of the present invention to provide a method of directional skin tightening comprising:
(i) producing a plurality of excised tissue portions in a region of skin tissue;
(ii) affixing to the skin region a stretching/compression device, having at least two portions, adapted to provide contraction or expansion of said skin region in at least one predetermined direction; and,
(iii) securing at least one portion of said stretching/compression device to said skin;
(iv) applying tension therebetween said two portions, thereby providing directional skin tightening in said skin tissue.

It is another object of the present invention to provide the method as defined above, wherein said step of securing at least one portion of said stretching/compression device to said skin provides contraction of said skin region in said predetermined direction.

It is another object of the present invention to provide the method as defined above, additionally comprising step of securing the second portion of said stretching/compression device to said skin.

It is another object of the present invention to provide the method as defined above, wherein said step of applying tension therebetween said two portions additionally comprising step of securing the second portion of said stretching/compression device to said skin and pulling one portion relative to the other.

It is another object of the present invention to provide the method as defined above, wherein said stretching/compression device comprising a long and short portion; wherein said short portion comprises at least one buckle-like element having at least one slot hole therewithin; further wherein said long portion is adapted to be in physical communication with said short portion by threading thereof through said at least one slot hole and securing the same to said short portion.

It is another object of the present invention to provide the method as defined above, wherein said long portion comprises at least one adhesive layer adapted to secure attachment of said short portion and said long portion.

It is another object of the present invention to provide the method as defined above, wherein said short portion comprises at least one adhesive layer adapted to secure attachment of said short portion and said long portion.

It is another object of the present invention to provide the method as defined above, wherein said long portion comprises Velcro adapted to secure attachment of said short portion and said long portion.

It is another object of the present invention to provide the method as defined above, wherein said short portion comprises Velcro adapted to secure attachment of said short portion and said long portion.

It is another object of the present invention to provide the method as defined above, wherein said step of applying tension therebetween said two portions applies force in the range of 20N/mm2-40N/mm2.

It is another object of the present invention to provide the method as defined above, wherein said tension applied in said step of applying tension therebetween said two portions is adjustable based on at least one parameter selected from a group consisting of skin type, age of the patient, type of treatment and any combination thereof.

It is another object of the present invention to provide the method as defined above, wherein said step of applying tension therebetween said two portions is performed at a direction selected from a group consisting of the x-, y-, and/or z-direction and any combination thereof with respect to said stretching/compression device and said skin to provide said directional tightening.

It is another object of the present invention to provide the method as defined above, wherein said stretching/compression device comprises at least one occlusion layer adapted to control humidity and/or promote wound healing of said skin.

It is another object of the present invention to provide the method as defined above, wherein said stretching/compression device comprises at least one absorption layer adapted to absorb wound exudate.

It is another object of the present invention to provide the method as defined above, wherein said stretching/compression device is provided in a skin tone color or is transparent or semi-transparent.

It is another object of the present invention to provide the method as defined above, wherein said step of producing a plurality of excised tissue portions in a region of skin tissue is performed by a system comprising at least one robotic arm, said at least one robotic arm comprising at least one skin coring instrument.

It is another object of the present invention to provide the method as defined above, wherein said at least one skin coring instrument comprising at least one punch configured to contact a surface of the skin to generate holes in the skin tissue by excising portions of the skin tissue.

It is another object of the present invention to provide the method as defined above, wherein said at least one punch is at least 3 punches.

It is another object of the present invention to provide the method as defined above, wherein said skin coring instrument comprises:
a micro-coring punch including at least six micro-coring needles;
a mechanism configured to rotate each of the micro-coring needles around at least one axis of symmetry of each needle and wherein rotation of each of the micro-coring needles synchronized with the rotation of the rest of the micro-coring needles;
a mechanism configured to advance the micro-coring punch towards skin and penetrate the skin to a depth of at least two millimeters; and
a mechanism configured to step a micro-coring punch and locate the micro-coring punch such that two facets of a stepped micro-coring punch hexagon cross two facets of a first micro-coring punch hexagon.

It is another object of the present invention to provide the method as defined above, wherein the micro-coring punch is attached to a computer-controlled robotic arm capable of moving in six or more axes (degrees of freedom).

It is another object of the present invention to provide the method as defined above, wherein the computer-controlled robotic arm manipulates the micro-coring punch including five micro-coring needles.

It is another object of the present invention to provide the method as defined above, further comprising a video camera configured to provide visual feedback of at least the micro-coring punch and the skin and a closed-loop force sensor to determine when the punches break the skin.

It is another object of the present invention to provide the method as defined above, wherein the five micro-coring needles in vertices of the pentagonal pattern.

It is another object of the present invention to provide the method as defined above, wherein an area between two intersecting facets of the first hexagon and two facets of a stepped hexagon forms a rhomb (i.e., a rhombus).

It is another object of the present invention to provide the method as defined above, wherein the rhomb contains at least two micro-coring needles located at opposite vertices of the rhomb.

It is another object of the present invention to provide the method as defined above, wherein at least one vortex of the stepped hexagon, is located on an inscribed circle with a diameter equal to half of the diameter of the hexagon.

It is another object of the present invention to provide the method as defined above, wherein rotation of each of the micro-coring needles synchronized with the rotation of the rest of the micro-coring needles.

It is another object of the present invention to provide the method as defined above, wherein the mechanism configured to synchronize the rotation of each of the micro-coring needles with the rotation of the rest of the micro-coring needles, is one of a group of mechanisms consisting of gears or friction belts.

It is another object of the present invention to provide the method as defined above, wherein the micro-coring punch advances towards the skin and penetrates the skin to a depth of at least two millimeters.

It is another object of the present invention to provide the method as defined above, wherein the mechanism configured to advance the micro-coring punch towards the skin and penetrate the skin is one of a group of mechanisms consisting of a robotic arm or a screw.

It is another object of the present invention to provide the method as defined above, wherein at least a portion of said at least one punch is disposable.

It is another object of the present invention to provide the method as defined above, wherein said at least one punch is adapted to penetrate said skin either in a simultaneously or sequentially manner.

It is another object of the present invention to provide the method as defined above, wherein said at least one punch is characterized by either a similar or substantially different cross section area.

It is another object of the present invention to provide the method as defined above, wherein said at least one punch is adapted to penetrate said skin to a depth of 1 to 4 mm.

It is another object of the present invention to provide the method as defined above, wherein at least a portion of said at least one punch is characterized by a radius of 0.15 mm-1.0 mm, namely 0.6-0.75 mm.

It is another object of the present invention to provide the method as defined above, wherein said cross section area is selected from a group consisting of circular, rectangular, triangular, hexagonal, oval, staggered rows, parallel rows, a spiral pattern, a square or rectangular pattern, a radial distribution and any combination thereof.

It is another object of the present invention to provide the method as defined above, wherein said system additionally comprising at least one controller adapted to control the positioning of said at least one robotic arm relatively to said skin area.

It is another object of the present invention to provide the method as defined above, wherein said controller comprising at least one engine adapted to control at least one parameter selected from a group consisting of the rotation, translation, angle of penetration of said at least one robotic arm relatively to said skin, depth of penetration, coverage rate, the diameter of at least one excised tissue multiplied by number of cores, different area of said skin to be treated and any combination thereof.

It is another object of the present invention to provide the method as defined above, wherein said parameters are adjusted manually by the operator or automatically by said controller.

It is another object of the present invention to provide the method as defined above, wherein said parameters are real time adjusted.

It is another object of the present invention to provide the method as defined above, wherein said rotation is at a speed in the range of 1000-7000 RPM, namely 3000-7000 RPM.

It is another object of the present invention to provide the method as defined above, wherein said translation is at a speed in the range of 0-500 mm/sec.

It is another object of the present invention to provide the method as defined above, wherein said translation of said at least one robotic arm relatively to said skin changes as said at least one robotic arm gets closer to said skin.

It is another object of the present invention to provide the method as defined above, wherein said rotation of said at least one robotic arm changes as said at least one robotic arm gets closer to said skin and penetrates said skin.

It is another object of the present invention to provide the method as defined above, wherein each punch of said at least one punch rotates individually in a predefined direction. It is emphasized that it could be that each of the punches could rotated in a different direction.

It is another object of the present invention to provide the method as defined above, wherein all of said punches rotate simultaneously.

It is another object of the present invention to provide the method as defined above, wherein each punch of said at least one punch translates individually.

It is another object of the present invention to provide the method as defined above, wherein all of said punches translate simultaneously.

It is another object of the present invention to provide the method as defined above, wherein said controller comprising stopping mechanism adapted to limit the depth to which said at least one punch is penetrate said skin.

It is another object of the present invention to provide the method as defined above, wherein said angle of penetration is substantially perpendicular to said skin.

It is another object of the present invention to provide the method as defined above, wherein said controller is adapted to define at least one no-fly zone; said no-fly zone is defined as an area to which said system provides no treatment.

It is another object of the present invention to provide the method as defined above, wherein said system additionally provide the skin with additives.

It is another object of the present invention to provide the method as defined above, wherein said additives are selected from a group consisting of therapeutic agents, saline solution growth factors, platelet-derived growth factor (PDGF), transforming growth factor beta (TGF-B), fibroblast growth factor (FGF), epidermal growth factor (EGF), and keratinocyte growth factor); one or more stem cells; steroids, agents which prevent post-inflammatory skin hyperpigmentation, hydroquinone, azelaic acid, kojic acid, mandelic acid, or niacinamide; one or more analgesics; one or more antifungals; one or more anti-inflammatory agents, or a mineralocorticoid agent, an immune selective anti-inflammatory derivative; one or more antimicrobials; a foam; or a hydrogel, one or more antiseptics, one or more antiproliferative agents, one or more emollients; one or more hemostatic agents, a procoagulant, an anti-fibrinolytic agent, one or more procoagulative, one or more anticoagulative agents, one or more immune modulators, including corticosteroids and non-steroidal immune modulators, one or more proteins; or one or more vitamins and any combination thereof.

It is another object of the present invention to provide the method as defined above, wherein said system additionally comprising at least one imaging subsystem adapted to guide said at least one skin coring instrument.

It is another object of the present invention to provide the method as defined above, wherein said imaging subsystem comprises at least one selected from a group consisting at least one camera, under skin imaging such as ultrasound-based imaging, OCT and any combination thereof.

It is another object of the present invention to provide the method as defined above, wherein said system additionally comprising at least one vacuum subsystem adapted to apply suction to remove excising portions of said skin tissue.

It is another object of the present invention to provide the method as defined above, wherein said skin could be part of a treatment area selected from a group consisting of forehead, cheeks, jaw line, neck, upper arms, tummy, abdomen, face, eyelid, nose, forehead, chin, forehead, lips, nose, neck, thighs, chest, legs, back and any combination thereof.

It is another object of the present invention to provide the method as defined above, wherein said method is used for focal elimination of redundant dermal tissue for skin tightening, at least partially scar removal, skin rejuvenation, at least partially removal of pigment, at least partially tattoo removal, veins, acne, allodynia, blemishes, ectopic dermatitis, hyperpigmentation, hyperplasia, lentigo or keratosis, loss of translucency, loss of elasticity, melasma, photodamage, psoriasis, rhytides, wrinkles, sallow color, scar contracture, scarring, wrinkles, folds, acne scars, dyschromia, striae, surgical scars, cellulite, tattoos removal, cheek wrinkles, facial wrinkles, facial folds, skin aging, skin contraction, skin irritation/sensitivity, skin laxity, striae, vascular lesions, angioma, erythema, hemangioma, papule, port wine stain, rosacea, reticular vein, or telangiectasia, or any other unwanted skin irregularities and any combination thereof.

It is another object of the present invention to provide the method as defined above, wherein said system utilizes at least one selected from a group consisting of mechanical visualization, OCT, Ultrasound to image the treated area and at least one selected from a group consisting of machine learning algorithms, artificial intelligence, image processing and any combination thereof to efficiency select the preferred location of the tissue to be treated to enhance outcome of said treatment.

It is another object of the present invention to provide the method as defined above, wherein an areal fraction of excised tissue portions is less than about 70% of the skin region.

It is another object of the present invention to provide the method as defined above, wherein an areal fraction of excised tissue portions is less than about 10% of the skin region.

It is another object of the present invention to provide the method as defined above, wherein an areal fraction of excised tissue portions is in the range of about 5% to about 30% of the skin region.

It is another object of the present invention to provide the method as defined above, comprising pre-stretching the skin region before producing the plurality of excised tissue.

It is another object of the present invention to provide a method of dermal micro-coring, comprising:
providing a micro-coring punch including six micro-coring needles;
applying to at least one segment of skin a micro-coring punch and performing at least one micro-coring process;
wherein each consecutive a micro-coring punch is locating the micro-coring punch such that at least one facet of a stepped punch hexagon cross one facet of a previous hexagon.

It is another object of the present invention to provide the method as defined above, wherein each consecutive a micro-coring punch is locating the micro-coring punch such that at least two facets of a stepped punch hexagon cross two facets of a previous hexagon.

It is another object of the present invention to provide the method as defined above, wherein each micro-coring punch applied to said at least one segment of the skin includes stepping the micro-coring punch in at least one of X and Y directions.

It is another object of the present invention to provide the method as defined above, wherein each consecutive a micro-coring punch includes stepping the micro-coring punch on a distance equal at least to the radius of a circle in which the hexagon pattern is inscribed.

It is another object of the present invention to provide the method as defined above, wherein the stepping a micro-coring punch on a distance equal to the diameter of a circle in which the hexagon pattern is inscribed forms a plurality of hexagons with a radius twice the original hexagon radius.

It is another object of the present invention to provide the method as defined above, wherein locating the five micro-coring needles in vertices of the pentagonal pattern.

It is another object of the present invention to provide the method as defined above, wherein an area between the two intersecting facets of the first hexagon and two facets of a stepped hexagon form a rhomb.

It is another object of the present invention to provide the method as defined above, wherein the rhomb contains at least two micro-coring needles located at opposite vertices of the rhomb.

It is another object of the present invention to provide the method as defined above, wherein locating at least one vortex of the stepped hexagon, on an inscribed into the hexagon circle with a diameter equal to half of the diameter of the hexagon.

It is another object of the present invention to provide the method as defined above, wherein following the skin coring applying a positive pressure (squeezing) and compressing circular holes to get optimal skin healing and aesthetic skin tightening result.

It is another object of the present invention to provide the method as defined above, wherein said step of rotating each of the micro-coring needles is around at least one axis of symmetry.

It is another object of the present invention to provide the method as defined above, wherein synchronizing the rotation of each of the micro-coring needles with the rotation of the rest of the micro-coring needles.

It is another object of the present invention to provide the method as defined above, wherein advancing the micro-coring punch towards the skin, and penetrating the skin to a depth of at least two millimeters.

It is another object of the present invention to provide the method as defined above, wherein applying vacuum and puling dissected skin cores through tubing into a disposal canister.

It is another object of the present invention to provide the method as defined above, wherein flushing the tubing by a liquid to remove clogs in the tubing.

It is another object of the present invention to provide the method as defined above, wherein aligning the punches perpendicular to the skin.

It is another object of the present invention to provide the method as defined above, wherein using a closed-loop force sensor and visual feedback to determine when the punches break the skin.

It is another object of the present invention to provide the method as defined above, wherein retracting the punches and moving the punches to a next treatment location.

It is another object of the present invention to provide an apparatus for dermal micro-coring, comprising:
a micro-coring punch including six micro-coring needles;
a mechanism configured to rotate each of the micro-coring needles around at least one axis of symmetry of each needle and wherein rotation of each of the micro-coring needles synchronized with the rotation of the rest of the micro-coring needles;
a mechanism configured to advance the micro-coring punch towards the skin and penetrate the skin to a depth of at least two millimeters; and
a mechanism configured to step a micro-coring punch and locate the micro-coring punch such that one facet of a stepped micro-coring punch hexagon cross one facet of a first micro-coring punch hexagon.

It is another object of the present invention to provide the apparatus as defined above, wherein said mechanism configured to step a micro-coring punch and locate the micro-coring punch such that two facets of a stepped micro-coring punch hexagon cross two facets of a first micro-coring punch hexagon

It is another object of the present invention to provide the apparatus as defined above, wherein the micro-coring punch is attached to a computer-controlled robotic arm capable of moving in six or more axes (degrees of freedom).

It is another object of the present invention to provide the apparatus as defined above, wherein the computer-controlled robotic arm manipulates the micro-coring punch (including six micro-coring needles).

It is another object of the present invention to provide the apparatus as defined above, wherein six micro-coring needles are utilized.

It is another object of the present invention to provide the apparatus as defined above, wherein rotation of each of the micro-coring needles synchronized with the rotation of the rest of the micro-coring needles.

It is another object of the present invention to provide the apparatus as defined above, wherein a mechanism configured to synchronize the rotation of each of the micro-coring needles with the rotation of the rest of the micro-coring needles, is one of a group of mechanisms consisting of gears or friction belts.

It is another object of the present invention to provide the apparatus as defined above, wherein the step of the micro-coring punch is equal at least to half of the radius of a circle into which the hexagon is inscribed.

It is another object of the present invention to provide the apparatus as defined above, wherein an area between two intersecting (crossing) facets of the first hexagon and two facets of a stepped hexagon form a rhomb.

It is another object of the present invention to provide the apparatus as defined above, wherein the rhomb contains at least two micro-coring needles located at opposite vertices of the rhomb.

It is another object of the present invention to provide the apparatus as defined above, wherein at least one vortex of the stepped hexagon, is located on an inscribed circle with a diameter equal to half of the diameter of the hexagon.

It is another object of the present invention to provide the apparatus as defined above, further comprising a video camera configured to provide visual feedback of at least the micro-coring punch and the skin and a closed-loop force sensor to determine when the punches break the skin.

It is another object of the present invention to provide the apparatus as defined above, wherein the micro-coring punch advances towards the skin, and penetrates the skin to a depth of at least two millimeters.

It is another object of the present invention to provide the apparatus as defined above, wherein a mechanism configured to advance the micro-coring punch towards the skin and penetrate the skin, is one of a group of mechanisms consisting of a robotic arm or a screw.

It is another object of the present invention to provide the apparatus as defined above, wherein the mechanism configured to synchronize the rotation of each of the micro-coring needles with the rotation of the rest of the micro-coring needles, is one of a group of mechanisms consisting of gears or friction belts.

It is another object of the present invention to provide a method of increasing density of dermal micro-coring holes, comprising:
providing a micro-coring punch including five micro-coring needles arranged in a pentagonal pattern centered around a sixth micro-coring needle;
applying to a segment of skin said micro-coring punch and performing a first micro-coring operation;
stepping said micro-coring punch to treat the second segment of skin; and
wherein each next step of a micro-coring punch is locating the micro-coring punch such that vortices of the second and subsequent hexagons are located on an inscribed circle with a diameter equal to half of the diameter of the hexagon.

It is another object of the present invention to provide the method as defined above, wherein locating each second and subsequent hexagons of the micro-coring punch such that two facets of the second and subsequent hexagons cross two facets of the previous hexagon.

It is another object of the present invention to provide the method as defined above, wherein the distance between two neighbor corings is half of the radius of the hexagon.

It is another object of the present invention to provide the method as defined above, wherein locating a second micro-coring punch (larger than the first punch) coaxial with the first punch.

It is another object of the present invention to provide an apparatus for dermal micro-coring of a segment of skin area and directional tightening thereof, comprising:
a micro-coring punch including at least one micro-coring needle, preferably five arranged in a pentagonal pattern centered around a sixth micro-coring needle;
a mechanism configured to rotate each of the micro-coring needles around at least one axis of symmetry of each needle and wherein rotation of each of the micro-coring needles synchronized with the rotation of the rest of the micro-coring needles;
a mechanism configured to advance the micro-coring punch towards skin and penetrate the skin to a depth of at least two millimeters; and
a mechanism configured to step a micro-coring punch and locate the micro-coring punch such that two facets of a stepped micro-coring punch hexagon cross two facets of a first micro-coring punch hexagon.

It is another object of the present invention to provide the apparatus as defined above, wherein said at least one micro-coring needle is five micro-coring needles.

It is another object of the present invention to provide the apparatus as defined above, wherein the micro-coring punch is attached to a computer-controlled robotic arm capable of moving in six or more axes (degrees of freedom).

It is another object of the present invention to provide the apparatus as defined above, wherein the computer-controlled robotic arm manipulates the micro-coring punch including six micro-coring needles.

It is another object of the present invention to provide the apparatus as defined above, further comprising a video camera configured to provide visual feedback of at least the micro-coring punch and the skin and a closed-loop force sensor to determine when the punches break the skin.

It is another object of the present invention to provide the apparatus as defined above, wherein the five micro-coring needles in vertices of the pentagonal pattern.

It is another object of the present invention to provide the apparatus as defined above, wherein an area between two intersecting facets of the first hexagon and two facets of a stepped hexagon form a rhomb.

It is another object of the present invention to provide the apparatus as defined above, wherein the rhomb contains at least two micro-coring needles located at opposite vertices of the rhomb.

It is another object of the present invention to provide the apparatus as defined above, wherein at least one vortex of the stepped hexagon, is located on an inscribed circle with a diameter equal to half of the diameter of the hexagon.

It is another object of the present invention to provide the apparatus as defined above, wherein rotation of each of the micro-coring needles synchronized with the rotation of the rest of the micro-coring needles.

It is another object of the present invention to provide the apparatus as defined above, wherein the mechanism configured to synchronize the rotation of each of the micro-coring needles with the rotation of the rest of the micro-coring needles, is one of a group of mechanisms consisting of gears or friction belts.

It is another object of the present invention to provide the apparatus as defined above, wherein the micro-coring punch advances towards the skin and penetrates the skin to a depth of at least two millimeters.

It is still an object of the present invention to provide the apparatus as defined above, wherein the mechanism configured to advance the micro-coring punch towards the skin and penetrate the skin is one of a group of mechanisms consisting of a robotic arm or a screw.

It is another object of the present invention to provide a method of directional skin tightening of a skin region, comprising:
(i) producing a plurality of excised tissue portions in a region of skin tissue; and,
(ii) applying energy to said skin region to provide contraction or expansion of said skin region in a predetermined direction; thereby providing directional skin tightening in said skin tissue.

It is another object of the present invention to provide the method as defined above, additionally comprising step of applying stretching tension to said skin region before said step of producing a plurality of excised tissue portions.

It is another object of the present invention to provide the method as defined above, wherein said directional skin tightening is performed at a direction selected from a group consisting of x-, y-, and/or z-direction and any combination thereof.

It is another object of the present invention to provide the method as defined above, wherein said step of producing a plurality of excised tissue portions in a region of skin tissue is performed by means selected from a group consisting of mechanical means, application of temperature to heat and evacuate tissue, application of laser, RF, coblation, coagulation, microwave energy, ultrasound and any other application of energy and any combination thereof.

It is another object of the present invention to provide the method as defined above, wherein said step of applying energy to said skin region to provide contraction or expansion of said skin region is performed by means selected from a group consisting of mechanical means, application of temperature to heat and evacuate tissue, application of laser, RF, coblation, coagulation, microwave energy, ultrasound, and any other application of energy and any combination thereof.

It is another object of the present invention to provide the method as defined above, wherein said step of producing a plurality of excised tissue portions in a region of skin tissue is performed by a system comprising at least one robotic arm, said at least one robotic arm comprising at least one skin coring instrument.

It is another object of the present invention to provide the method as defined above, wherein said at least one skin coring instrument comprising a at least one punch is configured to contact a surface of the skin to generate holes in the skin tissue by excising portions of the skin tissue.

It is another object of the present invention to provide the method as defined above, wherein said at least one punch is at least 6 punches; 5 of which are disposed at a pentagonal shape around a sixth central punch.

It is another object of the present invention to provide the method as defined above, wherein at least a portion of said at least one punch is disposable.

It is another object of the present invention to provide the method as defined above, wherein said all of said punches are adapted to penetrate said skin either in a simultaneously or sequentially manner.

It is another object of the present invention to provide the method as defined above, wherein all of said punches are characterized by either a similar or substantially different cross section area.

It is another object of the present invention to provide the method as defined above, wherein said plurality of punches are adapted to penetrate said skin to a depth of 1 to 4 mm.

It is another object of the present invention to provide the method as defined above, wherein at least one of said punches is characterized by a radius of 0.15 mm-1.0 mm.

It is another object of the present invention to provide the method as defined above, wherein said cross section area is selected from a group consisting of circular, rectangular, triangular, hexagonal, oval, staggered rows, parallel rows, a spiral pattern, a square or rectangular pattern, a radial distribution and any combination thereof.

It is another object of the present invention to provide the method as defined above, wherein said system additionally comprising at least one controller adapted to control the positioning of said at least one robotic arm relatively to said skin area.

It is another object of the present invention to provide the method as defined above, wherein said controller comprising at least one engine adapted to control at least one parameter selected from a group consisting of the rotation, translation, angle of penetration of said at least one robotic arm relatively to said skin, depth of penetration, coverage rate, the diameter of at least one excised tissue multiplied by number of cores, different area of said skin to be treated and any combination thereof.

It is another object of the present invention to provide the method as defined above, wherein said parameters are adjusted manually by the operator or automatically by said controller.

It is another object of the present invention to provide the method as defined above, wherein said parameters are real time adjusted.

It is another object of the present invention to provide the method as defined above, wherein said rotation is at a speed in the range of 1000-7000 RPM.

It is another object of the present invention to provide the method as defined above, wherein said translation is at a speed in the range of 0-500 mm/sec.

It is another object of the present invention to provide the method as defined above, wherein said translation of said at least one robotic arm relatively to said skin changes as said at least one robotic arm gets closer to said skin.

It is another object of the present invention to provide the method as defined above, wherein said rotation of said at least one robotic arm changes as said at least one robotic arm gets closer to said skin and penetrates said skin.

It is another object of the present invention to provide the method as defined above, wherein at least one punch rotates individually in a predefined direction in a predetermined speed.

It is another object of the present invention to provide the method as defined above, wherein all of said punches rotate simultaneously.

It is another object of the present invention to provide the method as defined above, wherein each punch translates individually.

It is another object of the present invention to provide the method as defined above, wherein all of said punches translate simultaneously.

It is another object of the present invention to provide the method as defined above, wherein said controller comprising stopping mechanism adapted to limit the depth to which at least one punch penetrates said skin.

It is another object of the present invention to provide the method as defined above, wherein said angle of penetration is substantially perpendicular to said skin.

It is another object of the present invention to provide the method as defined above, wherein said controller is adapted to define at least one no-fly zone; said no-fly zone is defined as an area to which said system provides no treatment.

It is another object of the present invention to provide the method as defined above, wherein said system additionally provide the skin with additives.

It is another object of the present invention to provide the method as defined above, wherein said additives are selected from a group consisting of therapeutic agents, saline solution growth factors, platelet-derived growth factor (PDGF), transforming growth factor beta (TGF-ß), fibroblast growth factor (FGF), epidermal growth factor (EGF), and keratinocyte growth factor); one or more stem cells; steroids, agents which prevent post-inflammatory skin hyperpigmentation, hydroquinone, azelaic acid, kojic acid, mandelic acid, or niacinamide; one or more analgesics; one or more antifungals; one or more anti-inflammatory agents, or a mineralocorticoid agent, an immune selective anti-inflammatory derivative; one or more antimicrobials; a foam; or a hydrogel, one or more antiseptics, one or more antiproliferative agents, one or more emollients; one or more hemostatic agents, a procoagulant, an anti-fibrinolytic agent, one or more procoagulative, one or more anticoagulative agents, one or more immune modulators, including corticosteroids and non-steroidal immune modulators, one or more proteins; or one or more vitamins and any combination thereof.

It is another object of the present invention to provide the method as defined above, wherein said system additionally comprising at least one imaging subsystem adapted to guide said at least one skin coring instrument.

It is another object of the present invention to provide the method as defined above, wherein said imaging subsystem comprises at least one selected from a group consisting at least one camera, under skin imaging such as ultrasound-based imaging, OCT and any combination thereof.

It is another object of the present invention to provide the method as defined above, wherein said system additionally comprising at least one vacuum subsystem adapted to apply suction to remove excising portions of said skin tissue.

It is another object of the present invention to provide the method as defined above, wherein said skin could be part of a treatment area selected from a group consisting of forehead, cheeks, jaw line, neck, upper arms, tummy, abdomen, face, eyelid, nose, forehead, chin, forehead, lips, nose, neck, thighs, chest, legs, back and any combination thereof.

It is another object of the present invention to provide the method as defined above, wherein said method is used for focal elimination of redundant dermal tissue for skin tightening, at least partially scar removal, skin rejuvenation, at least partially removal of pigment, at least partially tattoo removal, veins, acne, allodynia, blemishes, ectopic dermatitis, hyperpigmentation, hyperplasia, lentigo or keratosis, loss of translucency, loss of elasticity, melasma, photodamage, psoriasis, rhytides, wrinkles, sallow color, scar contracture, scarring, wrinkles, folds, acne scars, dyschromia, striae, surgical scars, cellulite, tattoos removal, cheek wrinkles, facial wrinkles, facial folds, skin aging, skin contraction, skin irritation/sensitivity, skin laxity, striae, vascular lesions, angioma, erythema, hemangioma, papule, port wine stain, rosacea, reticular vein, or telangiectasia, or any other unwanted skin irregularities and any combination thereof.

It is another object of the present invention to provide the method as defined above, wherein said system utilizes at least one selected from a group consisting of mechanical visualization, OCT, Ultrasound, machine learning algorithms, artificial intelligence, image processing and any combination thereof to efficiency select the preferred location of the tissue to be treated to enhance outcome of said treatment.

It is another object of the present invention to provide the method as defined above, wherein an areal fraction of excised tissue portions is in the range of about 5% to about 30% of the skin region.

It is another object of the present invention to provide the method as defined above, wherein an areal fraction of excised tissue portions is less than about 10% of the skin region.

It is another object of the present invention to provide the method as defined above, comprising pre-stretching the skin region before producing the plurality of excised tissue.

It is another object of the present invention to provide a system of directional skin tightening of a skin region, comprising:
(i) means for producing a plurality of excised tissue portions in a region of skin tissue; and,
(ii) means for applying at least one type of energy to said skin region to provide contraction or expansion of said skin region in a predetermined direction, so as to provide directional skin tightening in said skin tissue.

It is another object of the present invention to provide the system as defined above, additionally comprising means for applying stretching tension to said skin region before said step of producing a plurality of excised tissue portions.

It is another object of the present invention to provide the system as defined above, wherein said directional skin tightening is performed at a direction selected from a group consisting of x-, y-, and/or z-direction and any combination thereof.

It is another object of the present invention to provide the system as defined above, wherein said means of producing a plurality of excised tissue portions in a region of skin tissue comprising means selected from a group consisting of mechanical means, application of temperature to heat and evacuate tissue, application of laser, RF, coblation, coagulation, microwave energy, ultrasound, application of any other type of energy and any combination thereof.

It is another object of the present invention to provide the system as defined above, wherein said means of applying energy to said skin region to provide contraction or expansion of said skin region comprising means selected from a group consisting of mechanical means, application of temperature to heat and evacuate tissue, application of laser, RF, coblation, coagulation, microwave energy, ultrasound, application of any other type of energy and any combination thereof.

It is another object of the present invention to provide the system as defined above, wherein said means of producing a plurality of excised tissue portions in a region of skin tissue comprising a system comprising at least one robotic arm, said at least one robotic arm comprising at least one skin coring instrument.

It is another object of the present invention to provide the system as defined above, wherein said at least one skin coring instrument comprising a plurality of punches (or at least one punch), configured to contact a surface of the skin to generate holes in the skin tissue by excising portions of the skin tissue.

It is another object of the present invention to provide the system as defined above, wherein said plurality of punches are at least 6 punches; 5 of which are disposed at a pentagonal shape around a sixth central punch.

It is another object of the present invention to provide the system as defined above, wherein at least a portion of said plurality of punches are disposable.

It is another object of the present invention to provide the system as defined above, wherein said plurality of punches are adapted to penetrate said skin either in a simultaneously or sequentially manner.

It is another object of the present invention to provide the system as defined above, wherein said plurality of punches are characterized by either a similar or substantially different cross section area.

It is another object of the present invention to provide the system as defined above, wherein said plurality of punches are adapted to penetrate said skin to a depth of 1 to 4 mm.

It is another object of the present invention to provide the system as defined above, wherein at least a portion of said plurality of punches are characterized by a radius of 0.15 mm-1.0 mm.

It is another object of the present invention to provide the system as defined above, wherein said cross section area is selected from a group consisting of circular, rectangular, triangular, hexagonal, oval, staggered rows, parallel rows, a spiral pattern, a square or rectangular pattern, a radial distribution and any combination thereof.

It is another object of the present invention to provide the system as defined above, wherein said system additionally comprising at least one controller adapted to control the positioning of said at least one robotic arm relatively to said skin area.

It is another object of the present invention to provide the system as defined above, wherein said controller comprising at least one engine adapted to control at least one parameter selected from a group consisting of the rotation, translation, angle of penetration of said at least one robotic arm relatively to said skin, depth of penetration, coverage rate, the diameter of at least one excised tissue multiplied by number of cores, different area of said skin to be treated and any combination thereof.

It is another object of the present invention to provide the system as defined above, wherein said parameters are adjusted manually by the operator or automatically by said controller.

It is another object of the present invention to provide the system as defined above, wherein said parameters are real time adjusted.

It is another object of the present invention to provide the system as defined above, wherein said rotation is at a speed in the range of 1000-7000 RPM.

It is another object of the present invention to provide the system as defined above, wherein said translation is at a speed in the range of 0-500 mm/sec.

It is another object of the present invention to provide the system as defined above, wherein said translation of said at least one robotic arm relatively to said skin changes as said at least one robotic arm gets closer to said skin.

It is another object of the present invention to provide the system as defined above, wherein said rotation of said at least one robotic arm changes as said at least one robotic arm gets closer to said skin and penetrates said skin.

It is another object of the present invention to provide the system as defined above, wherein each punch of said plurality of punches rotates individually in a predefined direction in a predetermined speed.

It is another object of the present invention to provide the system as defined above, wherein said plurality of punches rotate simultaneously.

It is another object of the present invention to provide the system as defined above, wherein each punch of said plurality of punches translates individually.

It is another object of the present invention to provide the system as defined above, wherein said plurality of punches translate simultaneously.

It is another object of the present invention to provide the system as defined above, wherein said controller comprising stopping mechanism adapted to limit the depth to which at least a portion of said plurality of punches penetrate said skin.

It is another object of the present invention to provide the system as defined above, wherein said angle of penetration is substantially perpendicular to said skin.

It is another object of the present invention to provide the system as defined above, wherein said controller is adapted to define at least one no-fly zone; said no-fly zone is defined as an area to which said system provides no treatment.

It is another object of the present invention to provide the system as defined above, wherein said system additionally provide the skin with additives.

It is another object of the present invention to provide the system as defined above, wherein said additives are selected from a group consisting of therapeutic agents, saline solution growth factors, platelet-derived growth factor (PDGF), transforming growth factor beta (TGF-B), fibroblast growth factor (FGF), epidermal growth factor (EGF), and keratinocyte growth factor); one or more stem cells; steroids, agents which prevent post-inflammatory skin hyperpigmentation, hydroquinone, azelaic acid, kojic acid, mandelic acid, or niacinamide; one or more analgesics; one or more antifungals; one or more anti-inflammatory agents, or a mineralocorticoid agent, an immune selective anti-inflammatory derivative; one or more antimicrobials; a foam; or a hydrogel, one or more antiseptics, one or more antiproliferative agents, one or more emollients; one or more hemostatic agents, a procoagulant, an anti-fibrinolytic agent, one or more procoagulative, one or more anticoagulative agents, one or more immune modulators, including corticosteroids and non-steroidal immune modulators, one or more proteins; or one or more vitamins and any combination thereof.

It is another object of the present invention to provide the system as defined above, wherein said system additionally comprising at least one imaging subsystem adapted to guide said at least one skin coring instrument.

It is another object of the present invention to provide the system as defined above, wherein said imaging subsystem comprises at least one selected from a group consisting at least one camera, under skin imaging such as ultrasound-based imaging, OCT and any combination thereof.

It is another object of the present invention to provide the system as defined above, wherein said system additionally comprising at least one vacuum subsystem adapted to apply suction to remove excising portions of said skin tissue.

It is another object of the present invention to provide the system as defined above, wherein said skin could be part of a treatment area selected from a group consisting of forehead, cheeks, jaw line, neck, upper arms, tummy, abdomen, face, eyelid, nose, forehead, chin, forehead, lips, nose, neck, thighs, chest, legs, back and any combination thereof.

It is another object of the present invention to provide the system as defined above, wherein said system is used for focal elimination of redundant dermal tissue for skin tightening, at least partially scar removal, skin rejuvenation, at least partially removal of pigment, at least partially tattoo removal, veins, acne, allodynia, blemishes, ectopic dermatitis, hyperpigmentation, hyperplasia, lentigo or keratosis, loss of translucency, loss of elasticity, melasma, photodamage, psoriasis, rhytides, wrinkles, sallow color, scar contracture, scarring, wrinkles, folds, acne scars, dyschromia, striae, surgical scars, cellulite, tattoos removal, cheek wrinkles, facial wrinkles, facial folds, skin aging, skin contraction, skin irritation/sensitivity, skin laxity, striae, vascular lesions, angioma, erythema, hemangioma, papule, port wine stain, rosacea, reticular vein, or telangiectasia, or any other unwanted skin irregularities and any combination thereof.

It is another object of the present invention to provide the system as defined above, wherein said system utilizes at least one selected from a group consisting of mechanical visualization, OCT, Ultrasound, machine learning algorithms, artificial intelligence, image processing and any combination thereof to efficiency select the preferred location of the tissue to be treated to enhance outcome of said treatment.

It is another object of the present invention to provide the system as defined above, wherein an areal fraction of excised tissue portions is in the range of about 5% to about 30% of the skin region.

It is another object of the present invention to provide the system as defined above, wherein an areal fraction of excised tissue portions is less than about 10% of the skin region.

It is another object of the present invention to provide the system as defined above, comprising pre-stretching the skin region before producing the plurality of excised tissue.

It is another object of the present invention to provide the method as defined above, additionally comprising step of providing the system with at least one cutting element adapted to grind said excised tissue so as to facilitate extraction thereof.

It is another object of the present invention to provide the method as defined above, additionally comprising step of providing the system with at least one cutting element adapted to grind said excised tissue so as to facilitate extraction thereof.

It is another object of the present invention to provide the method as defined above, additionally comprising step of providing the system with at least one cutting element adapted to grind said excised tissue so as to facilitate extraction thereof.

It is another object of the present invention to provide the method as defined above, additionally comprising step of providing the system with at least one cutting element adapted to grind said excised tissue so as to facilitate extraction thereof.

It is another object of the present invention to provide the method as defined above, additionally comprising step of providing the system with at least one cutting element adapted to grind said excised tissue so as to facilitate extraction thereof.

It is another object of the present invention to provide the apparatus as defined above, additionally comprising at least one cutting element, integrated within said skin coring instrument, adapted to grind said excised tissue so as to facilitate extraction thereof.

It is another object of the present invention to provide the apparatus as defined above, additionally comprising at least one cutting element, integrated within said skin coring instrument, adapted to grind said excised tissue so as to facilitate extraction thereof.

It is another object of the present invention to provide the apparatus as defined above, additionally comprising at least one cutting element, integrated within said skin coring instrument, adapted to grind said excised tissue so as to facilitate extraction thereof.

It is another object of the present invention to provide the system as defined above, additionally comprising at least one cutting element, integrated within said skin coring instrument, adapted to grind said excised tissue so as to facilitate extraction thereof.

It is another object of the present invention to provide the method as defined above, wherein said at least one skin coring instrument is in communication with at least one RF generator, adapted to apply RF energy to the skin and tissue, so as to fractional ablate/coagulate the tissue. It is another object of the present invention to provide the method as defined above, wherein said application of RF energy is either simultaneously or sequentially with the coring of said skin.

It is another object of the present invention to provide the method as defined above, wherein said at least one skin coring instrument is in communication with at least one pulsed electromagnetic frequency generator.

It is another object of the present invention to provide the method as defined above, wherein said pulsed electromagnetic frequency generator is adapted to provide at least one dynamic magnetic field pulses to said skin.

It is another object of the present invention to provide the method as defined above, wherein said dynamic magnetic field pulses are provided by means of at least one coil.

It is another object of the present invention to provide the method as defined above, wherein said at least one skin coring instrument is at least partially coiled by at least one coil.

It is another object of the present invention to provide the method as defined above, wherein said at least one skin coring instrument is adapted to simultaneously provide both said electromagnetic pulses and said RF energy to said skin.

It is another object of the present invention to provide the method as defined above, wherein said RF energy is provided in the shape of heat to said skin.

It is another object of the present invention to provide the method as defined above, wherein at least one of the following is being held true (a) the shape of said electromagnetic pulse is selected from the group consisting of square wave, a sine wave, a triangular wave, sawtooth wave, ramp waves, spiked wave or any combination thereof; (b) the magnetic field intensity B of each pulse applied by said pulsed electromagnetic frequency generator ranges between about 0 and about 3 Tesla; (c) the magnetic field intensity B of each pulse applied by said pulsed electromagnetic frequency generator ranges between about 0 to 40 Gauss; (d) the duration of each pulse applied by said pulsed electromagnetic frequency generator ranges between about 3 and about 1000 milliseconds; (e) the frequency F applied by the pulses of said pulsed electromagnetic frequency generator ranges between about 1 Hz and about 40 MHz; (f) the energy E applied by the pulses of said pulsed electromagnetic frequency generator ranges between about 1 and about 150 watts per pulse or any combination thereof; (g) the frequency F applied by the pulses applied by said step of applying pulsed electromagnetic therapy to said region to be higher than about 1 and lower than about 1 MHz; (h) the frequency F applied by said electromagnetic field pulses ranges between 1 Hz and 50 Hz; (i) the frequency of said RF energy ranges between 200 kHz and 40 MHz; (j) the power P applied by said RF energy pulses ranges between 1 W and 100 W of RMS average power; and any combination thereof.

It is another object of the present invention to provide the method as defined above, additionally comprising at least one temperature sensor.

It is another object of the present invention to provide the method as defined above, additionally comprising a mechanism for skin cooling, adapted to regulate the temperature of the skin.

It is another object of the present invention to provide the method as defined above, wherein the distal end of said at least one skin coring instrument additionally comprising at least one selected from a group consisting of at least one impedance, at least one temperature sensor and any combination thereof.

It is another object of the present invention to provide the method as defined above, wherein said at least one selected from a group consisting of at least one impedance, at least one temperature sensor and any combination thereof is adapted to provide indication as to the depth of penetration of each of said at least one skin coring instrument.

It is another object of the present invention to provide the method as defined above, wherein said at least one skin coring instrument additionally comprising at least one needle, adapted to inject at least one treatment substance to the treatment area.

It is another object of the present invention to provide the method as defined above, wherein said at least one treatment substance is selected from a group consisting of hyaluronic acid, botox, collagen, stem cells and any combination thereof.

It is another object of the present invention to provide an apparatus of fractional coring for directional skin tightening, comprising:
(i) means for producing a plurality of excised tissue portions in a region of skin tissue; and,
(ii) means for securing to the skin region a stretching/compression device, having at least two portions, adapted to provide contraction or expansion of said skin region in at least one predetermined direction;
thereby endorsing collagen growth and providing directional skin tightening in said skin tissue.

It is another object of the present invention to provide the apparatus as defined above, wherein said stretching/compression device comprising a long and short portion; wherein said short portion comprises at least one buckle-like element having at least one slot hole therewithin; further wherein said long portion is adapted to be in physical communication with said short portion by threading thereof through said at least one slot hole and securing the same to said short portion.

It is another object of the present invention to provide the apparatus as defined above, wherein said long portion comprises at least one adhesive layer adapted to secure attachment of said short portion and said long portion.

It is another object of the present invention to provide the apparatus as defined above, wherein said short portion comprises at least one adhesive layer adapted to secure attachment of said short portion and said long portion.

It is another object of the present invention to provide the apparatus as defined above, wherein said long portion comprises Velcro adapted to secure attachment of said short portion and said long portion.

It is another object of the present invention to provide the apparatus as defined above, wherein said short portion comprises Velcro adapted to secure attachment of said short portion and said long portion.

It is another object of the present invention to provide the apparatus as defined above, wherein said stretching/compression device comprises at least one occlusion layer adapted to control humidity and/or promote wound healing of said skin.

It is another object of the present invention to provide the apparatus as defined above, wherein said stretching/compression device comprises at least one absorption layer adapted to absorb wound exudate.

It is another object of the present invention to provide the apparatus as defined above, wherein said stretching/compression device is provided in a skin tone color or is transparent or semi-transparent.

It is another object of the present invention to provide the apparatus as defined above, wherein said producing a plurality of excised tissue portions in a region of skin tissue is performed by means selected from a group consisting of mechanical means, application of temperature to heat and evacuate tissue, application of laser, pulsed electromagnetic field, RF, coblation, coagulation, microwave energy, ultrasound, application of any other type of energy and any combination thereof.

It is another object of the present invention to provide the apparatus as defined above, wherein said producing a plurality of excised tissue portions in a region of skin tissue is performed by a system comprising at least one robotic arm, said at least one robotic arm comprising at least one skin coring instrument.

It is another object of the present invention to provide the apparatus as defined above, wherein said at least one skin coring instrument comprising at least one selected from a group consisting of at least one needle, at least one punch and any combination thereof; said at least one skin coring instrument is configured to contact a surface of the skin to generate holes in the skin tissue by excising portions of the skin tissue.

It is another object of the present invention to provide the apparatus as defined above, wherein said at least one said at least one skin coring instrument is at least 6 punches; 5 of which are disposed at a pentagonal shape around a sixth central punch.

It is another object of the present invention to provide the apparatus as defined above, wherein at least a portion of said at least one punch is disposable.

It is another object of the present invention to provide the apparatus as defined above, wherein at least two said at least one skin coring instrument are adapted to penetrate said skin either in a simultaneously or sequentially manner.

It is another object of the present invention to provide the apparatus as defined above, wherein at least two of said at least one skin coring instrument are characterized by either a similar or substantially different cross section area.

It is another object of the present invention to provide the apparatus as defined above, wherein said at least one skin coring instrument is adapted to penetrate said skin to a depth of 1 to 4 mm.

It is another object of the present invention to provide the apparatus as defined above, wherein said at least one skin coring instrument is characterized by a radius of 0.15 mm-1.0 mm.

It is another object of the present invention to provide the apparatus as defined above, wherein said cross section area is selected from a group consisting of circular, rectangular, triangular, hexagonal, oval, staggered rows, parallel rows, a spiral pattern, a square or rectangular pattern, a radial distribution and any combination thereof.

It is another object of the present invention to provide the apparatus as defined above, wherein said system additionally comprising at least one controller adapted to control the positioning of said at least one robotic arm relatively to said skin area.

It is another object of the present invention to provide the apparatus as defined above, wherein said controller comprising at least one engine adapted to control at least one parameter selected from a group consisting of the rotation, translation, angle of penetration of said at least one robotic arm relatively to said skin, depth of penetration, coverage rate, the diameter of at least one excised tissue multiplied by number of cores, different area of said skin to be treated and any combination thereof.

It is another object of the present invention to provide the apparatus as defined above, wherein said parameters are adjusted manually by the operator or automatically by said controller.

It is another object of the present invention to provide the apparatus as defined above, wherein said parameters are real time adjusted.

It is another object of the present invention to provide the apparatus as defined above, wherein said rotation is at a speed in the range of 1000-7000 RPM.

It is another object of the present invention to provide the apparatus as defined above, wherein said translation is at a speed in the range of 0-500 mm/sec.

It is another object of the present invention to provide the apparatus as defined above, wherein said translation of said at least one robotic arm relatively to said skin changes as said at least one robotic arm gets closer to said skin.

It is another object of the present invention to provide the apparatus as defined above, wherein said rotation of said at least one robotic arm changes as said at least one robotic arm gets closer to said skin and penetrates said skin.

It is another object of the present invention to provide the apparatus as defined above, wherein each of said at least one skin coring instrument rotates individually in a predefined direction in a predetermined speed.

It is another object of the present invention to provide the apparatus as defined above, wherein at least two of said skin coring instruments rotate simultaneously.

It is another object of the present invention to provide the apparatus as defined above, wherein each of said at least one skin coring instrument translates individually.

It is another object of the present invention to provide the apparatus as defined above, wherein at least two of said at least one skin coring instrument translate simultaneously.

It is another object of the present invention to provide the apparatus as defined above, wherein the distance between each pair of neighboring skin coring instrument can vary and be adjustable either before or during treatment.

It is another object of the present invention to provide the apparatus as defined above, wherein said controller comprising stopping mechanism adapted to limit the depth to which at least a portion of said at least one skin coring instrument penetrates said skin.

It is another object of the present invention to provide the apparatus as defined above, wherein said angle of penetration is substantially perpendicular to said skin.

It is another object of the present invention to provide the apparatus as defined above, wherein said controller is adapted to define at least one no-fly zone; said no-fly zone is defined as an area to which said system provides no treatment.

It is another object of the present invention to provide the apparatus as defined above, wherein said skin coring instrument comprising:

a micro-coring punch including at least five micro-coring needle arranged in a predetermined pattern centered around a sixth micro-coring needle;
a mechanism configured to rotate each of the micro-coring needles around at least one axis of symmetry of each needle and wherein rotation of each of the micro-coring needles synchronized with the rotation of the rest of the micro-coring needles;
a mechanism configured to advance the micro-coring punch towards skin and penetrate the skin to a depth of at least two millimeters; and
a mechanism configured to step a micro-coring punch and locate the micro-coring punch such that at least one element selected from a group consisting of vertex, facet and any combination thereof of a stepped micro-coring punch hexagon cross at least one element selected from a group consisting of vertex, facet and any combination thereof of a previous micro-coring punch hexagon.

It is another object of the present invention to provide the apparatus as defined above, wherein the micro-coring punch is attached to a computer-controlled robotic arm capable of moving in six or more axes (degrees of freedom).

It is another object of the present invention to provide the apparatus as defined above, wherein the computer-controlled robotic arm manipulates the micro-coring punch including five micro-coring needles.

It is another object of the present invention to provide the apparatus as defined above, further comprising a video camera configured to provide visual feedback of at least the micro-coring punch and the skin and a closed-loop force sensor to determine when the punches break the skin.

It is another object of the present invention to provide the apparatus as defined above, wherein the five micro-coring needles are in vertices of the pentagonal pattern.

It is another object of the present invention to provide the apparatus as defined above, wherein an area between two intersecting facets of the first hexagon and two facets of a stepped hexagon form a rhomb.

It is another object of the present invention to provide the apparatus as defined above, wherein the rhomb contains at least two micro-coring needles located at opposite vertices of the rhomb.

It is another object of the present invention to provide the apparatus as defined above, wherein at least one vortex of the stepped hexagon, is located on an inscribed circle with a diameter equal to half of the diameter of the hexagon.

It is another object of the present invention to provide the apparatus as defined above, wherein rotation of each of the micro-coring needles synchronized with the rotation of the rest of the micro-coring needles.

It is another object of the present invention to provide the apparatus as defined above, wherein the mechanism configured to synchronize the rotation of each of the micro-coring needles with the rotation of the rest of the micro-coring needles, is one of a group of mechanisms consisting of gears or friction belts.

It is another object of the present invention to provide the apparatus as defined above, wherein the micro-coring needles advance towards the skin and penetrates the skin to a depth of at least two millimeters.

It is another object of the present invention to provide the apparatus as defined above, wherein the mechanism configured to advance the micro-coring needles towards the skin and penetrate the skin is one of a group of mechanisms consisting of a robotic arm or a screw. Such advancement may be effectuated by a conveyor such as a belt, for example.

It is another object of the present invention to provide the apparatus as defined above, wherein said system additionally provide the skin with additives.

It is another object of the present invention to provide the apparatus as defined above, wherein said additives are selected from a group consisting of therapeutic agents, saline solution growth factors, platelet-derived growth factor (PDGF), transforming growth factor beta (TGF-B), fibroblast growth factor (FGF), epidermal growth factor (EGF), and keratinocyte growth factor); one or more stem cells; steroids, agents which prevent post-inflammatory skin hyperpigmentation, hydroquinone, azelaic acid, kojic acid, mandelic acid, or niacinamide; one or more analgesics; one or more antifungals; one or more anti-inflammatory agents, or a mineralocorticoid agent, an immune selective anti-inflammatory derivative; one or more antimicrobials; a foam; or a hydrogel, one or more antiseptics, one or more antiproliferative agents, one or more emollients; one or more hemostatic agents, a procoagulant, an anti-fibrinolytic agent, one or more procoagulative, one or more anticoagulative agents, one or more immune modulators, including corticosteroids and non-steroidal immune modulators, one or more proteins; or one or more vitamins and any combination thereof.

It is another object of the present invention to provide the apparatus as defined above, wherein said system additionally comprising at least one imaging subsystem adapted to guide said at least one skin coring instrument.

It is another object of the present invention to provide the apparatus as defined above, wherein said imaging subsystem comprises at least one selected from a group consisting at least one camera, under skin imaging such as ultrasound-based imaging, OCT and any combination thereof.

It is another object of the present invention to provide the apparatus as defined above, wherein said system additionally comprising at least one vacuum subsystem adapted to apply suction to remove excising portions of said skin tissue.

It is another object of the present invention to provide the apparatus as defined above, wherein said skin could be part of a treatment area selected from a group consisting of forehead, cheeks, jaw line, neck, upper arms, tummy, abdomen, face, eyelid, nose, forehead, chin, forehead, lips, nose, neck, thighs, chest, legs, back and any combination thereof.

It is another object of the present invention to provide the apparatus as defined above, wherein said apparatus is used for focal elimination of redundant dermal tissue for skin tightening, at least partially scar removal, skin rejuvenation, at least partially removal of pigment, at least partially tattoo removal, veins, acne, allodynia, blemishes, ectopic dermatitis, hyperpigmentation, hyperplasia, lentigo or keratosis, loss of translucency, loss of elasticity, melasma, photodamage, psoriasis, rhytides, wrinkles, sallow color, scar contracture, scarring, wrinkles, folds, acne scars, dyschromia, striae, surgical scars, cellulite, tattoos removal, cheek wrinkles, facial wrinkles, facial folds, skin aging, skin contraction, skin irritation/sensitivity, skin laxity, striae, vascular lesions, angioma, erythema, hemangioma, papule, port wine stain, rosacea, reticular vein, or telangiectasia, or any other unwanted skin irregularities and any combination thereof.

It is another object of the present invention to provide the apparatus as defined above, wherein said apparatus utilizes at least one selected from a group consisting of mechanical visualization, OCT, Ultrasound, machine learning algorithms, artificial intelligence, image processing and any combination thereof to efficiency select the preferred location of the tissue to be treated to enhance outcome of said treatment.

It is another object of the present invention to provide the apparatus as defined above, wherein an areal fraction of excised tissue portions is in the range of about 5% to about 30% of the skin region.

It is another object of the present invention to provide the apparatus as defined above, wherein an areal fraction of excised tissue portions is less than about 10% of the skin region.

It is another object of the present invention to provide the apparatus as defined above, additionally comprising at least one cutting element adapted to grind said excised tissue so as to facilitate extraction thereof.

It is another object of the present invention to provide the apparatus as defined above, wherein said at least one skin coring instrument is in communication with at least one RF generator, adapted to apply RF energy to the skin and tissue, so as to fractional ablate/coagulate the tissue.

It is another object of the present invention to provide the apparatus as defined above, wherein said application of RF energy is either simultaneously or sequentially with the coring of said skin.

It is another object of the present invention to provide the apparatus as defined above, wherein said at least one skin coring instrument is in communication with at least one pulsed electromagnetic frequency generator.

It is another object of the present invention to provide the apparatus as defined above, wherein said pulsed electromagnetic frequency generator is adapted to provide at least one dynamic magnetic field pulses to said skin.

It is another object of the present invention to provide the apparatus as defined above, wherein said dynamic magnetic field pulses are provided by means of at least one coil.

It is another object of the present invention to provide the apparatus as defined above, wherein said at least one skin coring instrument is at least partially coiled by at least one coil.

It is another object of the present invention to provide the apparatus as defined above, wherein said at least one skin coring instrument is adapted to simultaneously provide both said electromagnetic pulses and said RF energy to said skin.

It is another object of the present invention to provide the apparatus as defined above, wherein said RF energy is provided in the shape of heat to said skin.

It is another object of the present invention to provide the apparatus as defined above, wherein at least one of the following is being held true (a) the shape of said electromagnetic pulse is selected from the group consisting of square wave, a sine wave, a triangular wave, sawtooth wave, ramp waves, spiked wave or any combination thereof; (b) the magnetic field intensity B of each pulse applied by said pulsed electromagnetic frequency generator ranges between about 0 and about 3 Tesla; (c) the magnetic field intensity B of each pulse applied by said pulsed electromagnetic frequency generator ranges between about 0 to 40 Gauss; (d) the duration of each pulse applied by said pulsed electromagnetic frequency generator ranges between about 3 and about 1000 milliseconds; (e) the frequency F applied by the pulses of said pulsed electromagnetic frequency generator ranges between about 1 Hz and about 40 MHz; (f) the energy E applied by the pulses of said pulsed electromagnetic frequency generator ranges between about 1 and about 150 watts per pulse or any combination thereof; (g) the frequency F applied by the pulses applied by said step of applying pulsed electromagnetic therapy to said region to be higher than about 1 and lower than about 40M Hz; (h) the frequency F applied by said electromagnetic field pulses ranges between 1 Hz and 50 Hz; (i) the frequency of said RF energy pulses ranges between 200 kHz and 10 MHz; (j) the power P applied by said RF energy pulses ranges between 1 W and 100 W of RMS average power; and any combination thereof.

It is another object of the present invention to provide the apparatus as defined above, additionally comprising at least one temperature sensor.

It is another object of the present invention to provide the apparatus as defined above, additionally comprising a mechanism for skin cooling, adapted to regulate the temperature of the skin.

It is another object of the present invention to provide the apparatus as defined above, wherein the distal end of said at least one skin coring instrument additionally comprising at least one selected from a group consisting of at least one impedance, at least one temperature sensor and any combination thereof.

It is another object of the present invention to provide the apparatus as defined above, wherein said at least one selected from a group consisting of at least one impedance, at least one temperature sensor and any combination thereof is adapted to provide indication as to the depth of penetration of each of said at least one skin coring instrument.

It is another object of the present invention to provide the apparatus as defined above, wherein said at least one skin coring instrument additionally comprising at least one needle, adapted to inject at least one treatment substance to the treatment area.

It is another object of the present invention to provide the apparatus as defined above, wherein said at least one treatment substance is selected from a group consisting of hyaluronic acid, botox, collagen, stem cells and any combination thereof.

### BRIEF DESCRIPTION OF THE DRAWINGS

FIG. 1 illustrates the general operation of the device of the present invention.

FIG. 2 illustrates a dermal micro-coring process using a single punch.

FIGS. 3A, 3B, 3C, 3D and 3E illustrate two possible punch rotation drive types: belt driven and friction driven.

FIG. 4 illustrates the dissected skin cores from each punch are pulled up by vacuum.

FIGS. 5A-5B illustrate one arm, each of which utilizes 1 or more punches, as embodied in the system.

FIG. 6 illustrates a top views of the punches. The figures are drafted as coaxial punches.

FIGS. 7, 8 and 9 illustrate one instrument design configured to spread out punches allowing overlapping patterns.

FIGS. 10A-10B illustrate one embodiment of the stretching/compression device.

FIGS. 11-12 illustrate the short side, according to this embodiment, of the stretching/compression device.

FIGS. 13-14 illustrate the long side, according to this embodiment, of the stretching/compression device.

FIGS. 15A, 15B, 15C, 15D and 16 illustrate another embodiment of the directional tightening method and device according to the present invention.

FIG. 17 illustrates histological analysis-cross tissue sections after 0, 2 and 5 weeks post the fractional coring (tissue removal) treatment.

FIGS. 18A and 18B illustrate side and longitudinal views, respectively, of a biological unit removal tool having a movable retention member (retainer or retainer element) in the form of inner tines in a retracted or undeployed state.

FIGS. 19A and 19B illustrate side and longitudinal views of the biological unit removal tool of FIGS. 18A and 18B in a retentive state.

### DETAILED DESCRIPTION OF THE INVENTION

This invention relates to methods and devices for the tightening of skin and/or reduction of skin laxity by selectively opening or closing a plurality of small wounds formed by incision or excision of tissue. For example, tissue excision can be performed by fractional ablation of the epidermal and/or dermal layer of the skin with at least one hollow coring needle (or punch), by fractional laser ablation, by fractional radiofrequency (also refers to as RF) ablation, and/or by fractional ultrasonic ablation (using ultrasound). Various methods and devices are proposed to close the small wounds, including tunable or smart dressings that allow for titration of the tightening effect after application to the skin of a patient.

The device of the present invention excises a pattern of small dermal skin cores at desired density, and direction. Then, the remaining holes in the skin are closed, directionally, using manual compression methods such as compression tape or glue.

According to one embodiment of the present invention, the device of the present invention is designed for the removal of skin micro-cores in fractional manner—for different indications (e.g., skin resurfacing/wrinkle/lifting etc.).

According to one embodiment of the present invention, the coring mechanism is a single-use disposable cartridge consisting of at least one (preferably six (6)), up to 0.75 mm in diameter, hollow needles (or punches) inserted into the skin while rotating at about 7000 RPM with a maximum penetration depth of up to 3.5 mm to remove up to 15% of skin in the treatment area. This invention further relates to methods and devices for skin treatment. More, specifically, this invention relates to methods and devices for skin coring and tightening that would benefit from endorsing (e.g., promoting) collagen growth in a predetermined direction and providing directional skin tightening in said skin tissue thus, providing skin restoration or tightening. The device could be utilized in a wide variety of fields e.g., skin laxity, skin resurfacing, cheek wrinkles treatments, wrinkles treatments, folds treatments, acne scars removal, dyschromia treatment, striae treatment, surgical scars removal, cellulite treatment, tattoos removal and any combination thereof.

In particular embodiments, the present invention provides one or more of the following advantages. First, the methods and devices herein enable visualization of results in real time during the course of the treatment. One can envision asking the patient for feedback in real time during the treatment and adjusting the tightening to the patient preference. Second, the methods and devices herein are tunable, thereby allowing for titration of tightening after surgical hole or slit formation. For example, the tunable or smart dressings described herein allow adjustment of the tightening intensity, direction, and spatial distribution after the dressing has been applied or affixed to the patient's skin. In another example, titratable tightening can be achieved by selectively closing or opening a subset of slits or holes produced in an array. Third, the methods and devices herein requires less skill than that of a surgeon. One can envision treatment of patients in an outpatient setting, rather than requiring an inpatient, surgical setting. Fourth, the methods and devices herein constitute minimally invasive techniques, which can provide more predictable results and/or risk factors than that for more invasive techniques (e.g., plastic surgery) or non-invasive energy-based techniques (e.g., laser, coblation, coagulation, microwave energy, radiofrequency, or ultrasound). Fifth, the methods and devices herein can allow for less discriminate methods for treating the skin by forming holes or slits because the methods and devices allow for more discriminate control for closing such holes or slits. Sixth, the methods and devices herein can allow for rapid closing of holes or slits after treating the skin (e.g., within a few seconds after treating skin, such as within ten seconds), thereby minimizing the extent of bleeding and/or clotting within the holes or slits. Finally, the methods and devices herein can be useful for maximizing the tightening effect while minimizing healing time by optimizing tightening (e.g., by controlling the extent of skin pleating, such as by increasing the extent of skin pleating for some applications or skin regions and by decreasing the extent of skin pleating for other applications or skin regions, as described herein).

### Definitions

The term “about” refers hereinafter to +/−25% of any recited value.

The term “overlap” refers hereinafter to vertex, facet, cross sectional area and any combination thereof.

The term “Optical coherence tomography (OCT)” refers hereinafter to a non-invasive imaging. In other words, OCT is an imaging technique that uses low-coherence light to capture micrometer-resolution, two- and three-dimensional images from within optical scattering media (e.g., biological tissue). It is used for medical imaging and industrial nondestructive testing (NDT). Optical coherence tomography is based on low-coherence interferometry, typically employing near-infrared light. The use of relatively long wavelength light allows it to penetrate into the scattering medium. Confocal microscopy, another optical technique, typically penetrates less deeply into the sample but with higher resolution.

The term “mechanical visualization” refers hereinafter to either the use of ultrasound or OCT to image the under surface of the treated area skin/tissue. Such mechanical visualization is used to efficiency select the preferred location of the tissue to be treated to enhance outcome of said treatment. It should be noted that according to the present invention the term ‘mechanical visualization’ also includes cameras for imaging the surface of the treated area skin/tissue.

The term “incised” tissue portion or “incision” refers hereinafter to a cut, abrasion, or ablation of tissue, including a tissue portion in a skin region, or the act of cutting, abrading, destroying, or ablating tissue, a skin region, or one or more tissue portions. For example, an incision includes any cut, abrasion, or ablation into tissue, which can result in destruction of tissue or a portion thereof and, thereby, produce one or more holes or slits in the skin region. Exemplary methods of forming incised tissue portions or incisions include use of one or more blades, one or more solid needles, fractional laser ablation, fractional radiofrequency ablation, coblation, coagulation, microwave energy and/or fractional ultrasonic ablation, any useful tool for forming incisions, or any methods and apparatuses described herein.

The term “excised” tissue portion or “excision” refers hereinafter to a removed tissue, including a tissue portion from a skin region, or the act of removing tissue or one or more tissue portions from a skin region. Excision is usually referred to as “to surgically remove”. This term is often used in reference to removing a mass, excision means that tissue is removed, using a scalpel, laser, coblation, coagulation, ablation, ultrasound, microwave energy, RF, application of heat (to evaporate skin portions), mechanical applicator that ‘drills’ through the skin whilst suction is applies (during the drilling or thereafter) to removes the excised skin portion, or any another instrument. For example, an excision includes any removed tissue or tissue portion from a skin region, which can result in excised tissue portions having a particular geometry (e.g., a cylindrical geometry, rectangular, triangle etc. or any arbitrary shape) and produce one or more holes (i.e., negative space created by the removal of tissue) in the skin region. Exemplary methods of forming excised tissue portions or excisions include use of one or more hollow needles (optionally include one or more notches, extensions, protrusions, and/or barbs), one or more microaugers, one or more microabraders, any ablative means (including ablative lasers etc.)—may be used for incision and for excision, any useful tool for forming excisions, or any methods and apparatuses described herein.

The term “application of compression forces” refers hereinafter to a physical change in the compression tape (as disclosed hereafter). In this case, the forces applied are compression forces to compress the tape.

The term “application of expansion forces” refers hereinafter to a physical change in the compression tape (as disclosed hereafter). In this case, the forces applied are stretching forces to expand the tape.

The present invention features methods and devices to directionally tightening the skin after coring thereof (i.e., having one or more incised or excised tissue portions). In particular, exemplary devices include selectively opening or closing of holes and/or slits using a compression tape.

The device of the present invention is designed to enhance quality and productivity of skin laxity reduction procedures using advanced robotics, machine vision and software engineering.

The device implements dermal micro-coring approach to skin tightening. The device excises a pattern of predetermined small size dermal skin cores at desired density, and direction. The performed remaining holes in the skin are then closed, directionally, using manual compression methods such as compression tape or glue.

According to one embodiment of the present invention, the treatment parameters; i.e., desired density of the cores, depth, diameter etc. are automatically adjusted to the treated patient.

According to one embodiment, the density of the coring is 5-20% of the selected treated area. It is noted that according to another embodiment, the coverage rate (namely, the diameter of holes multiplied by number of holes) is 5-30% of the selected treated area.

The device may include the following elements:
1. At least one Robotic Arm and Controller that control the positioning of the arm relatively to the treated skin area.
2. Skin Coring Instrument and controls
3. RTC (real time controller) unit that includes at least one engine (e.g., a motor or robotic servo-motor) that controls the rotation, translation as well as the orientation of the robotic arm relatively to the treated skin area
4. Imaging Subsystem-to analyze treatment area and to guide the coring instrument.
5. Vacuum Subsystem-suction is applied to remove the excised tissue from the skin following the incision. Or alternatively a retention element (a retainer) is used that will hold the excised tissue, rendering the vacuum subsystem redundant. Hence, a vacuum is thus avoided by such embodiments and rendered unnecessary.
6. Stretching/compression device (e.g., compression tape) that will enable compression of the skin.

The skin coring instrument includes coring punches (e.g., the micro needles); either a single or multi-punch array for either simultaneous or sequentially coring the skin. It should be noted that the coring punches could be at least partially disposable.

According to one embodiment of the present invention, the coring instrument is a mechanical device that allows for small (0.4 to 1.0 mm), circular skin cores to be removed. According to another embodiment of the present invention any cross section (other than circular) is also within the scope of the present invention. E.g., circular, rectangular, triangular, hexagonal, oval, staggered rows, parallel rows, a spiral pattern, a square or rectangular pattern, a radial distribution and any combination thereof.

According to one embodiment, the coring instrument, has between 1 and 7 rotating (100-7000 RPM) coring punches that can be set to penetrate the skin surface and core to a depth of 1 to 4 mm. Suction is applied to remove the cores from the skin following the incision. The coring punches are disposable and a new one is used for each subject.

The coring element (e.g., the micro needles) has at least one sharp dermal punch to core out tissue (e.g., 0.25 mm-2.0 mm radius).

According to one embodiment, the dermal punches have a stopping mechanism (a stopper) to limit coring depth. A typical coring depth is configurable between 1 mm and 6 mm (and more specifically 1-4 mm) in steps of 0.5 mm.

According to one embodiment, the coring depth resolution is +/−0.1 mm.

According to one embodiment, each Individual punch is configured to rotate between 1000-7000 RPM.

According to one embodiment, each individual punch is able to translate into skin up to 500 mm/sec, preferably the translation speeds are less than 300 mm/sec.

According to one embodiment, each individual punch is configured to rotate at a speed that is less than 30 degree/sec.

According to one embodiment, the puncture angle is normal to the skin (+/−10 deg).

According to one embodiment, the mechanical extraction speed will be 1 cycle per second or faster.

According to one embodiment, the punch is flushed via saline solution. It should be noted that saline may be used via the punch to flush it between one coring step to the other but also to reduce friction of cored tissue and internal the part of the punch during cores evacuation.

The imaging subsystem is provided with illumination means (e.g., emitters such as LEDs) to illuminate the field of view of the imaging subsystem and to keep the cameras of the imaging subsystem exposure time at low latency.

The LED's wavelength is greater than 600 nm (warm white) to enable enough light to be reflected back from skin to cameras. Lower wavelengths tend to get absorbed more by human skin causing dark images.

The treated areas could be any of the body areas e.g., face, trunk, extremities, e.g., forehead, cheeks, jaw line, nose, forehead, neck, upper arms, thighs, abdomen, and tummy. According to another embodiment, the device of the present invention could be used for focal elimination of redundant dermal tissue for skin tightening, at least partially scar removal etc.

Post the coring process, the skin is tightened together by the stretching/compression device (as discussed hereinbelow) to promote healing thereof per the stretched/compressed tissue cores.

According to one embodiment, the stretching/compression device is adhesive based (e.g., surgical wound closure tape or glue). It is noted that the operator is able to compress skin in a different direction.

According to one embodiment of the present invention, the tensioning of the stretching/compression device, in order for it to effectively stretch the skin, has to be with pulling force of 20N/mm²-40N/mm².

It should be noted that according to one embodiment of the present invention, the operator can define in the treatment plan at least one of the following:
entering patient information into database
assigning surgery area and no-fly zones—where no treatment is provided to said area of skin tissue.
assigning areas with different densities
assigning areas with different hole pattern
assigning punch depth according to another embodiment of the present invention, adjustment of the treatment parameters could be enabled during treatment, in real-time; either manually, by the operator or automatically, by the system.

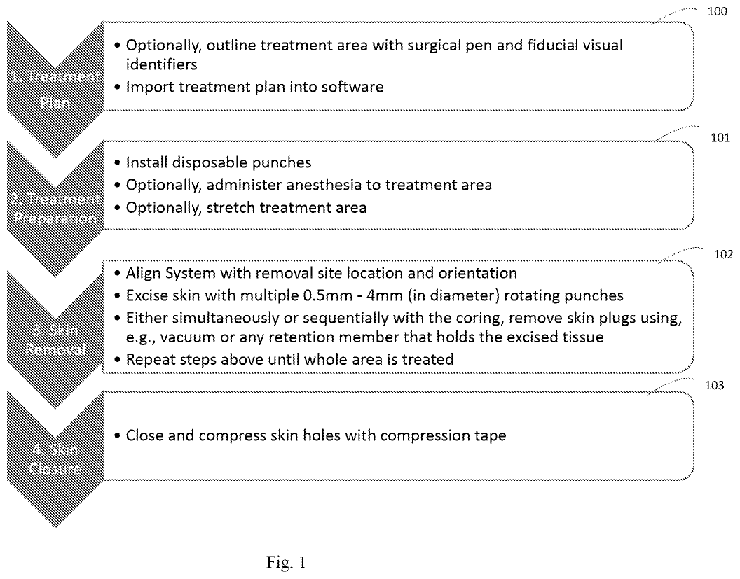

Reference is now being made to FIG. 1 which illustrates the general operation of the device of the present invention.

The first, optional step, step 100, is to outline the skin treatment area with surgical pen and/or adhesive biocompatible fiducial markers visual identifiers.

An image of treatment area with surgical lines and fiducial markers is enough for treatment planning software to automatically recognize and reconstruct treatment zone in 3D software environment. Within treatment planning software operator selects desired areas with skin removal density between 5%-30% and skin tightening direction. Thus, Once the treated area is outlined, the treatment plan is finalized (as disclosed hereinafter) and is loaded onto the system.

It should be noted that it is optional that the patient is administered with local anesthesia to avoid any pain during the procedure.

In case of treatment of folded skin, the operator may stretch the treatment area by applying adhesive stretch tapes. Adhesive tapes (e.g., Tegaderm) put skin under tension by pulling away in preferred directions. It should be noted that it is important to first stretch the skin and only then to excise tissue portions. Otherwise, the skin, due to its flexibility might be caught in the internal area within the drilling means (the punches and/or the needles). Thus, according to one embodiment, a method of directional skin tightening by fractional treatment is provided by the following steps:
(i) producing a plurality of excised tissue portions in a region of skin tissue; and,
(ii) securing at least one portion of a stretching/compression device (e.g., adhesive tapes Tegaderm), having at least two portions, to the skin region, adapted to provide contraction or expansion of said skin region in at least one predetermined direction;
(iii) applying tension therebetween said two portions thereby endorsing collagen growth and providing directional skin tightening in said region of skin tissue.

As disclosed above, in some cases (e.g., in case of loose skin), the step of securing said stretching/compression device to said skin region and application of tension (of either stretching or compression) to the skin is performed before said step of said producing a plurality of excised tissue portions in a region of skin tissue. This is to prevent any loosen skin being caught inside the drilling means (punches and/or needles).

According to another embodiment, the stretching/compression device is first stretched or compressed and only thereafter securing the second portion of said stretching/compression device to a different region of said skin.

According to another embodiment, the second portion of said stretching/compression device is secured to a different region of said skin.

According to another embodiment, applying tension therebetween said two portions additionally comprising step of securing the second portion of said stretching/compression device to said skin and pulling one portion relative to the other. As stated above, it is within the scope of the present invention when first the 2 portions of the stretching/compression device are secured to the skin, stretched and only thereafter the drilling means (the punches pr needles) provides a plurality of excised tissue portions.

It should be noted that even if the operator first applies tension therebetween the two portions of the stretching/compression device and only then produce a plurality of excised tissue portions in a region of skin tissue (while the tension is applied to the skin), it could very well be that the operator is required to apply additional tension therebetween the two portions of the stretching/compression device after the production of the excised tissue portions.

According to another embodiment of the present invention, it could be the said tension (stretching or compression) is applied simultaneously with the excision of skins portion by means of said drilling means (punches and/or needles).

The next step, step 101, is to install the disposable punches (and/or the needles) onto the device. The desired punches (and/or the needles) are selected pending. The desired density and depth of penetration.

Punches and/or the needles) are sharp, hollow and range from 0.4-4.0 mm in diameter. Larger hole may increase treatment speed but may not be appropriate for all skin types and body areas.

Optionally, a stoper is installed to limit maximum coring depth between 1-4 mm.

Next, step 102, the system is aligned with the area of the skin to be treated. Next, the skin is excised with multiple+/−0.4 to 4 mm (in diameter) punches (or needles).

According to one embodiment, the coring is performed by rotational movement of the punches (or needles), when the same are in contact with the skin. Alternatively, the coring is performed by rotational and translation movement of the punches (or needles).

Thereafter or simultaneously with the coring, the excised tissue is removed by means of vacuum. It should be noted that the system can utilize drilling means that evacuate the skin plugs along with the drilling and, therefore, vacuum means are not needed. In that case at least one retention element, integrated in the drilling means (the punches), is configured to hold the excised tissue (similarly to forceps), rendering the vacuum subsystem redundant. Thus, along with the drilling of the drilling means (the punches) performed into the skin, the retention element accumulates the excised skin plugs (tissue) and holds it. Thus, there will be no need for application of suction as the suction's main rule is to evacuate the excised skin plugs (tissue). In particular, the at least one retention implement may be implemented as a forceps-like device configured to exert pressure so as to hold the tissue.

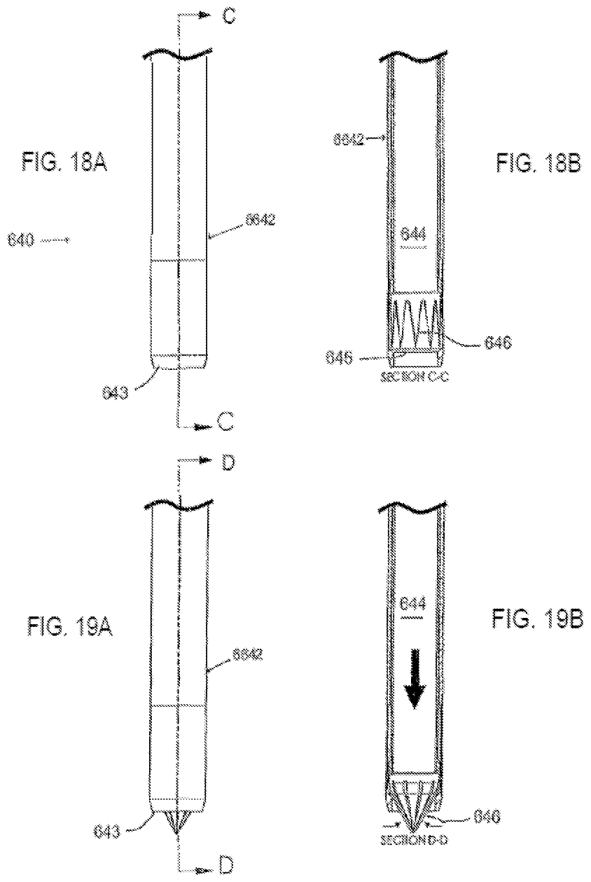

Exemplary implementations of the retention element are shown in FIGS. 18A and 18B, which depict side and longitudinal sectional views, respectively, of a biological unit removal tool having a movable retention member in the form of inner tines in a retracted or undeployed state. FIGS. 19A and 19B show the removal tool in a retention or deployed state. FIGS. 18A, 18B, 19A and 19D are exemplary depictions set forth in U.S. Pat. No. 8,696,686 issued Apr. 15, 2014, the entire contents of which are incorporated herein by reference, including for the apparatuses and methods disclosed therein. The exemplary removal tool 640 of FIGS. 18A, 18B, 19A and 19B has an outer tube or outer member 642 defining a lumen, and an inner tube or inner member 644 with a plurality of movable members or deformable tines 646 mounted on the inner tube. In the retracted position, the deformable tines 646 are flush with the inner diameter of the outer tube 642 and mounted to the distal end of the inner tube 644, which is allowed to move proximal/distal relative to the distal tip 643 of the outer tube. The distal tip 643 has a structure 645 that influences or guides the deformable tines to converge. The structure 645 is configured to assume the form of an inner ridge that guides the tines inward as the inner tube is advanced distally such that the tines converge. Alternatively, the structure may take the form of a taper, a step, an incline or any other form that guides the deformable tines to coapt. In the retention position, at least a portion of the retention member, e.g., the deformable tines, extend beyond the distal tip of the outer elongated member 642. The inner tube with tines may be made of various materials, including shape memory materials, for example, Nitinol, or Elgiloy, or cobalt chromium, or similar material which accommodates repetitive bending without fatigue (or with more tolerant fatigue properties), if desired, at the base of the tines. In some embodiments, the movable retention members need not be in the form of tines, but may be configured as thin wires, filaments, or paddle shaped structures for example, or varying shapes and surface finishes, and of various circumferential distributions.

The drilling means (the punches, microneedles) tools generally have a tubular elongated body with a cylindrical profile and a hollow lumen therethrough. According to one embodiment, at least one retention member described herein may be positioned not only at the distal portion of the drilling means, but also in various locations along the body of the drilling means, for example, a short distance from the distal end, or midway along the body of the drilling means, depending upon the configuration of the drilling means and its intended purpose. The terms “coupled,” or “attached,” or “connected,” or “mounted” as used herein, may mean directly or indirectly coupled, attached, integrated, or mounted through one or more intervening components.

A “retention member” as used herein refers to a structure, or a mechanism, or a number of structures and/or mechanisms that partially or fully retain a biological tissue in a lumen of the drilling means. The retention member may translate into or across the lumen, or radially constrict the lumen in a circumferential manner, for example, simply closing tightly about the tissue, located in the lumen to improve its retention and removal. The retention members described herein may be made of a variety of biocompatible materials, such as polypropylene, polyester, polyurethane, Teflon, Nitinol, stainless steel, etc. The configuration of the retention members may be solid, braided, filamentous, etc., and should not be considered limited to any one particular embodiment.

According to one embodiment the retention member may be movable along an axis of the drilling means (the punches). The retention member may form an integral part of the elongated body or may comprise a separate element attached within the lumen of the elongated body of the drilling means (the punches). In another version, the retention member comprises a portion made of a deformable material and the tool further comprises an actuation device adapted to deform at least the deformable portion of the retention member and constrict a lumen defined therein. For instance, the retention member comprises a plurality of portions made of deformable material, each two being separated by a spacer made of a substantially rigid material, such as Teflon, stainless steel, or titanium. The deformable material may be selected from the group consisting of silicone, rubber, gels, and fluids.

Another aspect of the invention is a biological tissue removal tool (that renders the use of suction redundant) comprising at least one movable retention member in communication with the drilling means (the punches). At least one of the drilling means (the punches) has a lumen sized to receive a biological specimen and a distal tip configured to penetrate a body surface. The retention member moves with respect to the drilling means (the punches) between a retracted position and a retention position in which the retention member is configured to project into or across the drilling means (the punches) proximally to the distal tip so as to impede movement in a distal tip direction of the biological specimen received in the lumen.

The retention member may be located and moveable from outside the drilling means (the punches) into the same. In one embodiment, the retention member is spring-biased, such as torsionally spring-biased, into the retention position. In another form, the retention member slides axially over the drilling means (the punches) between the retracted and retention positions and has a portion that passes into the drilling means (the punches) through an aperture in a wall of the elongated body in the retention position. For instance, the retention member may be a clip having at least two portions passing into the lumen through diametrically opposed apertures in the wall of the drilling means (the punches). In some alternatives, an actuator displaces the retention member between the retracted and retention positions, and the actuator may be automated. The retention member may be rotatable between the retracted and retention positions.

Another example of the at least one movable retention member is as follows. At least a portion of the retention member is axially movable over the drilling means (the punches) and the retention member is radially movable between a retracted position and a retention position, such that in the retention position at least a distal tip of the retention member extends beyond the distal tip of the drilling means (the punches) and converges.

It should be noted that the coring instrument could comprise several microneedles (punches) or a single one. It should be further noted that each of which could be independently operated or a sub-group thereof could be operated simultaneously. As stated above, before the coring step, the system aligns the punch(es) substantially perpendicular to the skin.

According to one embodiment there is provided at least one punch (or needle). Alternatively, at least 5 punches (or needles) are provided. The Punches (or needles) could rotate together, or each, individually. According to one embodiment all punches (or needles) are coupled to one common shaft operated by electric DC motor. According to another embodiment, there are multiple shafts operated by several electric DC motors.

According to one embodiment, the coring RPM is between 1000-7000 RPM.

As disclosed hereinafter the dissected skin cores from each punch/needle are pulled up by e.g., vacuum or any retention element(s) e.g., integrated within the punches, into accumulation chamber and eventually through tubing into canister for disposal. To ensure there are no clogs in tubing, liquid (e.g., saline) may be added to the chamber via a dripping mechanism to flush the system from at least one of the punch's end.

The vision subsystem, pointed at where punch tips will extend, detects 3D location of the skin surface and aligns punch(es) perpendicularly to the skin plane using moving arm joints. 3D Vision subsystem uses either passive (2 cameras) or active (2 cameras and infrared laser projector) stereo vision approach for sub millimeter accuracy.

Once aligned, the system translates rotating punch(es) to patient skin at high speed. Once the punch(es) approximate the skin they slow-down to a slower speed and then they will penetrate into the skin to 2-6 mm coring depth.

While inside the skin, the punch(es) use rotation sheer force to fracture and core out skin without compressing skin away from punch tips. Additionally, to avoid unnecessary skin compression, the system uses closed loop force sensor and vision feedback to determine when the punches break tougher epidermis layer and when the punches reach desired depth in dermis.

It is emphasized that, according to one embodiment of the present invention, before the treatment, a stretching element (e.g., Tegaderm) is used to stretch the affected skin or its surroundings before coring and thereby to stabilize the skin (so as to prevent compression before the coring).

At the end of the cycle, the system opens vacuum line to pull up and remove dermal tissue core. Next, the punch(es) are pulled back up above skin. Alternatively, the system may include at least one retention element adapted to hold or contain the extract excised tissue (without any applied vacuum).

According to one embodiment, the system can use automation and artificial intelligence algorithms to repeat and deliver described coring procedure according to the treatment plan rules. It should be noted that the artificial intelligence is used also to determine the treatment plan and coring protocol (e.g., the pattern of the coring elements).

Each coring cycle creates at least 1 hole; more preferable, 6 holes. Automation arranges and packs the holes patterns to achieve planned density.

By tracking unique fiducial identifiers system remembers where previous holes have been made therefore preventing possibility of overlapped holes. In addition, treatment automation deals with dynamic elements not captured in the treatment plan such as no-go zones, surgical equipment obstructions, bleeding etc.

The final step, step 103, is the directional tightening; in which the skin is compress, at the desired direction, by means of the compression tape (as disclosed hereinafter).

### The Treatment Plan

Before using the device of the present invention, an operator will outline the treatment area to be tightened on patient's skin. The operator marks treatment area using surgical pen and/or adhesive biocompatible fiducial markers.

An image of treatment area with surgical lines and fiducial markers is enough for treatment planning software to automatically recognize and reconstruct treatment zone in 3D software environment. Within treatment planning software operator selects desired areas with skin removal density between 5%-30% and skin tightening direction.

Depending on desired density and depth, the operator selects appropriate disposable punches. It should be noted that according to one embodiment of the present invention, the appropriate disposable punches are automatically recommended by the system (based on the treatment parameters; e.g., skin type, lesion to be treated, desired skin removal density etc.).

The punches (micro needles) are sharp, hollow and range from about +/−0.4-4.0 mm in diameter. Larger hole may increase treatment speed but may not be appropriate for all skin and lesion types. A typical coring depth would be between about 1 to about 4 mm.

The system of the present invention is positioned and orientated over patient skin either by operator manually, or automatically by finding treatment zone using vision subsystem. Vision system registers treatment zone with treatment plan by searching for particular fiducial identifiers or colored lines on the skin.

### The Coring Instrument and the Skin Removal Sub-System

Instrument performs dermal micro-coring process using multiple hollow rotating sharp punches. Each punch, shown on FIG. 2 has cylindrical shape with sharp conical cutting tip at the top. To ensure full dissection each punch has sharp inner edge and outside bevel. It should be noted that any other cross section area of the punch would work as well.

According to one embodiment of the present invention, there are X simultaneously rotating punches. X is in the range of 3-7 According to one embodiment, all punches rotate together and coupled to one common shaft operated by electric DC motor. According to another embodiment, each punch rotates individually and may or may not be coupled to one common shaft operated by electric DC motor.

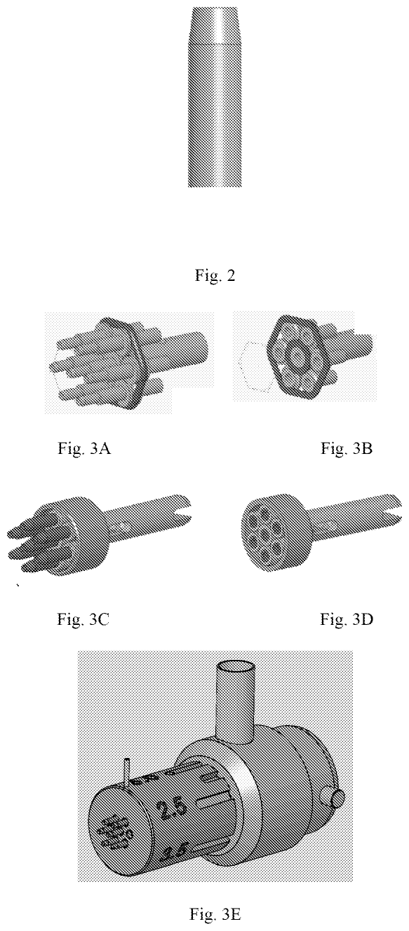

Reference is now made to FIGS. 3A-3D, illustrating the distal end of the applicator have 7 punches, 6 cerebralized around a 7th punch.

FIG. 3a-3d illustrate two possible punch rotation drive types: belt driven and friction driven. FIGS. 3a-3b illustrates the belt driven punch rotation type, before and after activation thereof, respectively. FIGS. 3c-3d illustrates the friction driven punch rotation type, before and after activation thereof, respectively.

Reference is now made to FIG. 3E, illustrating another embodiment of the distal end of the applicator have 6 punches (and not 7, as illustrated in FIGS. 3A-3D). As seen in the FIG. 3e, the six micro-coring needles (the punches) are arranged in 2 groups of 3 micro-coring needles, each arranged in vertices of a horizontally laying ‘V’ pattern. Namely, in a pattern of ‘>>’. It should be noted that it is within the scope of the present invention where the six micro-coring needles (the punches) are arranged in at least two horizontally lying ‘V’ shape, oppositely facing. Namely, in a of pattern ‘><’. However, one skilled in the art would appreciate that any pattern could be used. e.g., the pattern of the micro-coring needles (the punches) could be selected from a group consisting of a circular, hexagon, rectangular, square and any combination thereof.

In some embodiments, the coring RPM is between 1000-7000 RPM. Punches can translate together back and forth on a leadscrew or using robotic arm itself.

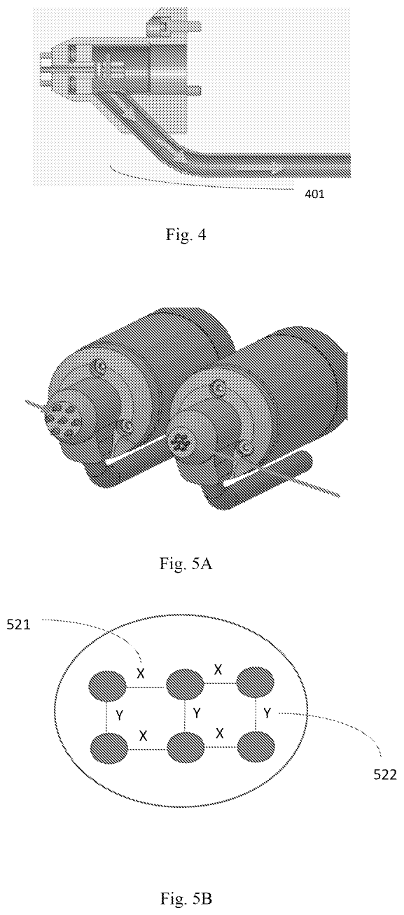

The punches are connected to skin core accumulation chamber. Dissected skin cores from each punch are pulled up by e.g., vacuum (see arrows 401) into accumulation chamber and eventually through tubing into canister (not shown) for disposal (see FIG. 4). It is noted that, as an alternative to the vacuum, the system may include at least one retention element adapted to hold or contain the extract excised tissue (without any applied vacuum). To ensure there are no clogs in tubing, liquid (e.g., saline) may be added to the chamber via a dripping mechanism to flush the system.

According to another embodiment, the liquid (e.g., saline) is added to reduce friction during the coring step.

According to one embodiment of the present invention, only one arm with 1 or more punch(es) is utilized in the system. According to another embodiment of the present invention, more than one arm, each of which utilizes 1 or more punch(es) is embodied in the system (as illustrated in FIG. 5a). In such an embodiment, each arm could utilize 1 or more punch(es) with the same properties (width, depth, cross section etc.) or alternatively, each arm would enclose one or more punch(es), each (or all) with individual/distinct properties.

According to another embodiment, each arm (and punches thereof) is characterized by different properties (e.g., width, depth, cross section of the punches, translation speed, rotation speed etc.).

According to another embodiment, all arms may include the same mechanism; alternatively, each arm comprises a different mechanism, e.g., different incision/excision means ((e.g., one arm makes an incision and the second arm is used for seeding or insertion/injection of additives, as disclosed hereinafter (e.g., threads, hyaluronic acid etc.)).

According to another embodiment of the present invention each punch is activated independently, such that in some embodiments, in the at least one arm of the device, there are several punches. However, each would be operated individually; thus, the operator may activate only a few of the punches and not all.

According to another embodiment of the present invention, the distance between each punch could be adjusted. Ref. is now made to FIG. 5b, which illustrates one arm 510 of the device having 6 punches 520, space apart at a distance X (see numerical ref. 521) and Y (see numerical ref. 522) from each other. According to one embodiment, said X and Y are adjustable such that the distances between the punches are changeable to better adjust thereof to the treatment.

### Automation and Artificial Intelligence Algorithms

According to one embodiment, the system uses automation and artificial intelligence algorithms to analyze the mechanical visualization input and to determine and establish the most appropriate coring pattern and plan. Thereafter, the artificial intelligence instructs to repeat and deliver described coring procedure according to the treatment plan rules.

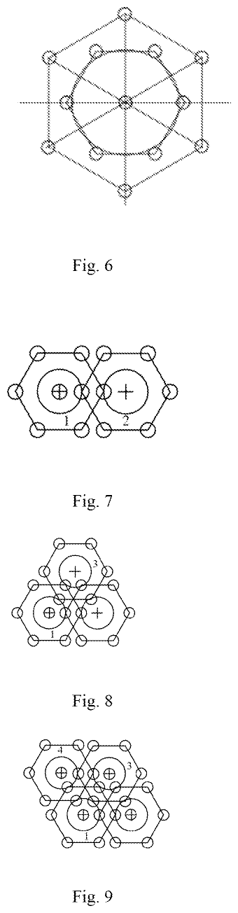

According to one embodiment, each coring cycle creates 6 holes arranged hexagonally (as illustrated in FIG. 6). Care should be given to the fact that there can be any number of punches. 6 is merely an example.

Automation arranges and packs hex patterns to achieve planned density. For example, on FIGS. 7-9, one instrument design may spread out punches allowing overlapping patterns, while another design may have punches packed tightly together. By tracking unique fiducial identifiers system remembers where previous holes have been made therefore preventing possibility of overlapped holes. In addition, treatment automation deals with dynamic elements not captured in the treatment plan such as no-go zones, surgical equipment obstructions, bleeding etc.

According to one embodiment, the overlapping patterns could have at least one point of excised tissue portion.

According to another embodiment, the device of the present invention also provides a mechanism configured to step a micro-coring punch and locate the micro-coring punch such that one element selected from a group consisting of vertex, facet and any combination thereof of a stepped micro-coring punch hexagon cross one element selected from a group consisting of vertex, facet and any combination thereof of a first micro-coring punch hexagon vertex, facet and any combination thereof of a first micro-coring punch hexagon. In some embodiments, a step mechanism is implemented as a stepper that translates the positions of the punches such that after a first coring session, the location of the punches is moved for a next coring session.

According to another embodiment, there can be an overlap between one coring step to the other (by vertex or facet); and, according to another embodiment of the present invention there can an overlap between consecutive coring steps (as can be seen in FIG. 7).

According to another embodiment of the present invention, the system utilizes artificial intelligence and/or mechanical visualization, OCT, Ultrasound, machine learning algorithms and/or image processing to provide inform decision as to the coring location. In other words, the system first scans the tissue to be treated and by means of at least one selected from a group consisting of artificial intelligence, mechanical visualization, OCT, Ultrasound, machine learning algorithms, image processing and any combination thereof, the system decides where it would be most beneficial to perform the coring.

### Directional Tightening

At the end of the treatment, the operator will use a stretching/compression device to close holes in the skin and promote healing per the new dimensions of the cored area, as employed by e.g., the compression.

According to one embodiment of the present invention, the stretching/compression device is an elastic compression tapes to close holes in the skin. Compressing skin together enables wound healing and collagen accumulation and adherence of the cored walls per its modified (compressed) configuration. Accordingly, with compression, cored holes are not as circles anymore, but ellipsoid and configured to be stabilized by new collagen in that position, promoting healing with the result of aesthetic skin tightening results due to the accumulated compressed cores per axis (with less chance of scars).

The stretching/compression device disclosed herein creates compression on the internal area and tension on the external area and eliminates unwanted puncture scars.

According to one embodiment of the present invention, the tension applied can be adjusted based on skin type to produce best results.

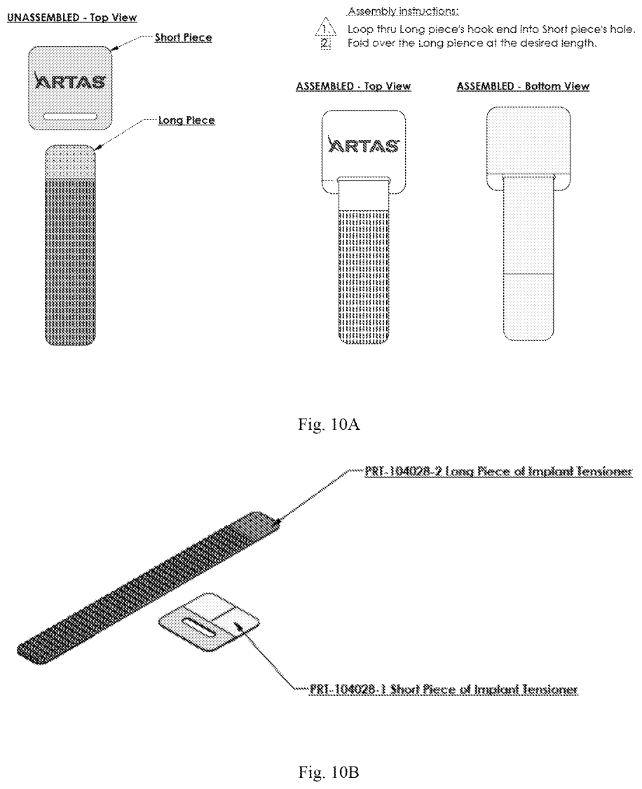

Reference is now made to FIGS. 10A-10B illustrating one embodiment of the stretching/compression device.

According to this embodiment of the present invention, the stretching/compression device has a long and short portion. The short portion comprises at least one buckle-like element having at least one slot hole therewithin. The long portion is adapted to be connected to the short side through said at least one slot hole therewithin. The long portion is threaded through said slot and secured to the short portion (as detailed hereinbelow). Said securement of said long portion to said short portion is by means of attaching at least one adhesive layer in said long portion to at least one adhesive layer in said short portion.

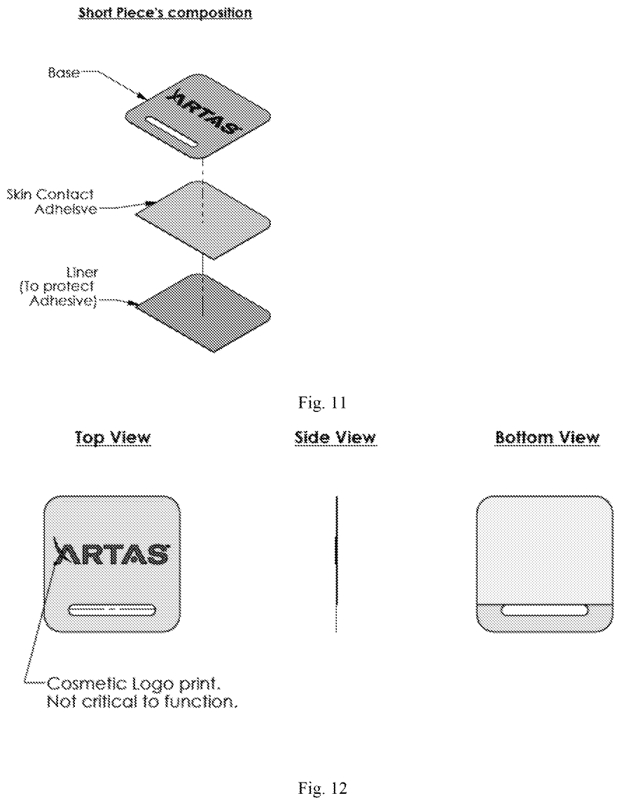

Reference is now made to FIGS. 11-12 illustrating the short side, according to this embodiment, of the stretching/compression device. According to this embodiment, the short side has base, adhesive, and liner.

The base can be made from any material that is strong enough to withstand, for example, 10PSI in shear force.

The adhesive can be made from any material that is strong enough to withstand, for example, 10PSI in shear force and the adhesive should adhere to skin well.

The liner is a cover that protects the adhesive until it is to be used.

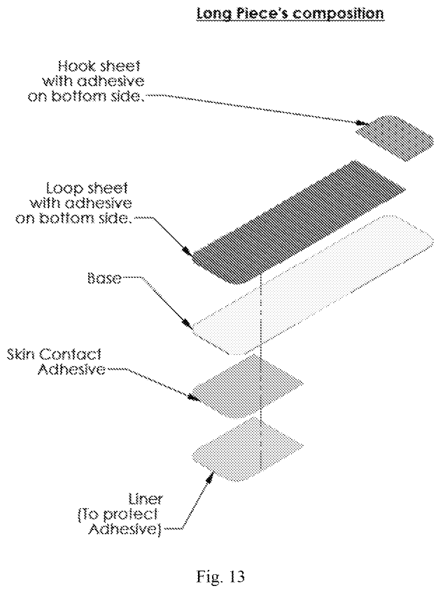

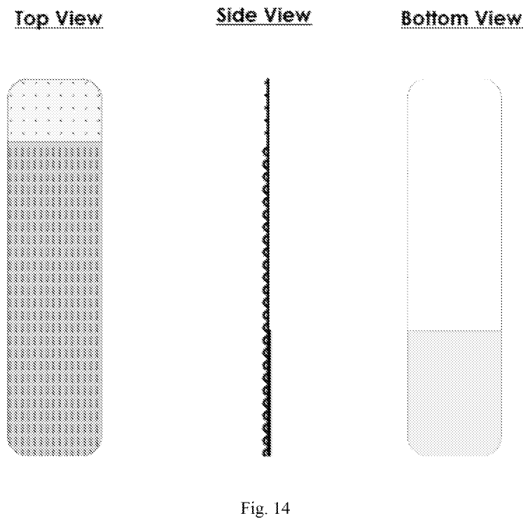

Reference is now made to FIGS. 13-14 illustrating the long side, according to this embodiment, of the stretching/compression device.

According to this embodiment, the long side has a base, an adhesive, a liner, and hook & loop sheets.

The base can be made from any material that is strong enough to withstand, for example, 10PSI in shear force.

The adhesive can be made from any material that is strong enough to withstand, for example, 10PSI in shear force and it should adhere to skin well.

The liner is a cover that protects the adhesive until it is to be used.

According to one embodiment, the hook and loop component (e.g., sheet) is Velcro. In an example, the hook sheet is the male side where it has tiny semi-rigid hooks on the top side and the loop sheet is the female side where it has thin loops on the top side. When the hook top side and loop top side come in contact with each other, the hooks hook onto the loops.

On the bottom side of both sheets, there is adhesive to allow the sheets to adhere to the base. This is not always necessary. An alternative is that the sheet acts as the base layer if the sheet is strong enough.

According to one embodiment, the loop sheet covers most of the long piece interface. This allows for smooth tape movement since the loop sheet may be thinner than the hook sheet. It is possible to reverse this; the hook sheet covers most of the long piece, but the hook sheet should be thin enough to be flexible enough to fold over (see side view note).

Once the stretching/compression device is placed over the holes in the skin, the operator stretches the same to create compression and/or tension to the desired level. Once the desired tension level is reached, the stretching/compression device can be closed and secured.

The application of the stretching/compression device will result in direction tightening of the skin.

The directionality of the skin region to which the stretching/compression device is applied, can also be optimized. In particular embodiments, the direction of skin tightening is determined by the directionality of the tensile force or compressive force being applied. It can be in the x-, y-, and/or z-direction with respect to the device or skin region.

The optimization of the applied tension of the stretching/compression device can provide numerous benefits. For instance, such tunability can allow real-time control of compressing and/or expanding the stretching/compression device after affixation thereof to the skin. This level of control can allow for personalized treatment of the patient based on the disease, disorder, or condition to be treated; the optimal cosmetic effect to be achieved; the optimal closure process to be achieved; and/or the timing and extent of the healing process observed for the particular patient. Furthermore, tunability can allow for less discriminate control over how the incisions or excisions in the skin region are made, as well as more discriminate control over selectively closing or opening the incisions or excisions.

The stretching/compression device can be affixed to the entire treated skin region or in a portion of the treated skin region. Directional or non-directional tightening can be achieved by producing a geometric arrangement of incisions and/or excisions that are treated similarly. Alternatively, such tightening can be achieved by a non-geometric arrangement of incisions and/or excisions in which only some of the incisions and/or excisions are opened or closed using the stretching/compression device.

It should be noted that when incision or excision occur-then wound healing process starts and, as commonly known, includes collagen synthesis and maturation. Thus, it is within the core of the present invention to facilitate its construction and accumulation per deformed cored area(s).

The tunable dressing can include an adhesive layer (e.g., formed from any adhesive material described herein). The adhesive layer can be continuous (i.e., a continuous layer of one or more adhesive materials attached to the proximal surface of a dressing) or discontinuous (i.e., a non-continuous layer of one or more adhesive materials attached to the proximal surface of a dressing). The adhesive layer can include any useful arrangement of the adhesive material. For instance, the adhesive layer can be tunable and allows for controlled compression or expansion.

In some embodiments, an adhesive layer includes a random, non-geometric, or geometric array of an adhesive material for tunability. In particular embodiments, the array allows for directional or non-directional compression and/or expansion as the dressing compresses and/or expands. In particular embodiments, the adhesive layer is discontinuous and includes an array of an adhesive material (e.g., an array of dots, where each dot gets closer together as the dressing compresses and each dot gets further apart as the dressing expands). Exemplary adhesive materials are described herein and include materials that promote collagen cross-linking, such as riboflavin or Rose Bengal, synthetic glues (e.g., cyanoacrylate, polyethylene glycol, or gelatin-resorcinol-formaldehyde), or biologic sealants (e.g., albumin-based or fibrin-based sealants that promote clotting).

The stretching/compression device can also include at least one occlusion layer (e.g., to control humidity and/or promote wound healing), at least one absorption layer (e.g., to absorb wound exudate), at least one reinforcement layer (e.g., to reinforce the layer and optionally formed from low-density polyethylene (LDPE), fluorinated ethylene propylene (FEP), or nylon), and/or at least one delivery layer (e.g., to delivery one or more therapeutic agents to enhance treatment thereof).

The stretching/compression device can be of any cosmetically appealing color, shape, and/or material. For example, the stretching/compression device can be provided in a skin tone color or is transparent or semi-transparent. Such transparent or semi-transparent dressings can additionally be helpful for visualization, e.g., for real-time tunability of the dressing and/or for affixing the stretching/compression device to the treated skin region.

According to another embodiment of the present invention, the stretching/compression device could either first be applied (i.e., secured) to skin (post excision of the skin portion) and only thereafter application of tension forces are applied thereto to provide the directional tightening of the skin.

According to another embodiment of the present invention, the stretching/compression device could either first be stretched and only then applied (i.e., secured) to skin (post excision of the skin portion). Once applied when the same is stretched the stretching/compression device (as it is elastic an dressing) would compress back to its original shape and hance apply compression tension to the skin thereto to provide the directional tightening of the skin.

In other words, the stretching/compression device could first go through a pretreatment, where stretching forces are applied thereto (for example by means of a dedicated device) and, once it is fully/partially stretched it is applied to the skin.

According to another embodiment of the present invention, the stretching/compression device that could be employed is simply an adhesive tape, e.g., 3M™ Tegaderm™, HP Transparent Film Dressing (see https://www.3 m.com/3M/en_US/company-us/all-3m-products/˜/3M-Tegaderm-HP-Transparent-Film-Dressing/?N=5002385+3293321973&rt=rud).

### Methods of Skin Tightening, More Specifically Direction Skin Tightening

The present invention relates to various methods and devices (e.g., the stretching/compression device) used to selectively open or close incisions and/or excisions (e.g., all or a portion of such incisions, such as microslits, and/or excisions, such as holes) formed in the skin region by the incised or excised tissue portions. The devices can be affixed to the entire treated skin region or in a portion of the treated skin region, which allow for directional or non-directional tightening by producing a geometric or non-geometric arrangement of incisions and/or excisions that are treated similarly or differently. Further, the devices can provide uniform or non-uniform compression and/or expression across the entire device or a portion thereof. Accordingly, these methods and devices can result in reducing the skin surface and/or tightening of the skin.

The methods can include contraction or expansion in one or more directions in at least a portion of the device (e.g., the dressing). The methods include, for example, affixing the stretching/compression device to a skin region having a plurality of incised tissue portions and/or excised tissue portions (e.g., where at least two of said tissue portions has at least one dimension that is less than about 1 mm or an areal dimension that is less than about 1 mm²). The device provides contraction or expansion of the skin region in one or more directions (e.g., in the x-, y-, z-, xy-, xz-, yz-, and/or xyz-directions, as described herein), where such contraction or expansion can be uniform or non-uniform. Furthermore, contraction or expansion arises by exposing an affixed device to one or more external stimuli (e.g., any described herein) that results of application of force (e.g., compression or stretching forces) on the stretching/compression device. In addition, such contraction and/or expansion can be adjusted after affixing the device. For example, after treating the skin and affixing the device, the device can be further expanded or to compress the skin region. In this manner, the device is tunable/adjustable.

The present invention also includes methods of tightening skin in a preferred direction (directional tightening of the skin (e.g., by compression and/or expansion exerted by the device)).

The present invention also includes optimizing the dimension of the incised or excised tissue portions to promote wound healing. Exemplary dimensions include circular and non-circular holes, such as elliptical holes. Non-circular holes can be formed by using an apparatus having a non-circular cross-section (e.g., a blade or a tube, such as a hollow tube, having a non-circular cross-section) or by pre-stretching the skin before treatment with an apparatus having a circular cross-section (e.g., a circular coring needle generates an elliptical hole in a non-stretched skin). In some embodiments, the long axis of the ellipse is perpendicular to the pre-stretching direction, where the elliptical hole can generate skin tightening preferentially in the direction of the short axis of the ellipse. Accordingly, the stretching/compression device can be affixed to a skin portion including one or more holes or one or more incised or excised tissue portions having one or more geometries.

It should be noted that when incision or excision occur-then wound healing process starts and, as commonly known, includes collagen synthesis and maturation. Thus, it is within the core of the present invention to facilitate its construction and accumulation per deformed cored area(s).

Adhesive Materials that can be Integrated in the Stretching/Compression Device.

An adhesive can be used within the dressing (e.g., as in the adhesive layer) or used in combination with any method described herein to promote skin tightening.

The adhesive can be a pressure-sensitive adhesive (PSA). The properties of pressure sensitive adhesives are governed by three parameters, tack (initial adhesion), peel strength (adhesion), and shear strength (cohesion). Pressure-sensitive adhesives can be synthesized in several ways, including solvent-borne, water-borne, and hot-melt methods. Tack is the initial adhesion under slight pressure and short dwell time and depends on the adhesive's ability to wet the contact surface. Peel strength is the force required to remove the PSA from the contact surface. The peel adhesion depends on many factors, including the tack, bonding history (e.g. force, dwell time), and adhesive composition. Shear strength is a measure of the adhesive's resistance to continuous stress. The shear strength is influenced by several parameters, including internal adhesion, cross-linking, and viscoelastic properties of the adhesive. Permanent adhesives are generally resistant to debonding and possess very high peel and shear strength.

Exemplary adhesives include a biocompatible matrix (e.g., those including at least one of collagen (e.g., a collagen sponge), low melting agarose (LMA), polylactic acid (PLA), and/or hyaluronic acid (e.g., hyaluranon); a photosensitizer (e.g., Rose Bengal, riboflavin-5-phosphate (R-5-P), methylene blue (MB), N-hydroxypyridine-2-(1H)-thione (N-HTP), a porphyrin, or a chlorin, as well as precursors thereof); a photochemical agent (e.g., 1,8 naphthalimide); a synthetic glue (e.g., a cyanoacrylate adhesive, a polyethylene glycol adhesive, or a gelatin-resorcinol-formaldehyde adhesive); or a biologic sealant (e.g., a mixture of riboflavin-5-phosphate and fibrinogen, a fibrin-based sealant, an albumin-based sealant, or a starch-based sealant). In particular embodiments, the adhesive is biodegradable.

Exemplary pressure-sensitive adhesives include natural rubber, synthetic rubber (e.g., styrene-butadiene and styrene-ethylene copolymers), polyvinyl ether, polyurethane, acrylic, silicones, and ethylene-vinyl acetate copolymers. A copolymer's adhesive properties can be altered by varying the composition (via monomer components) changing the glass transition temperature (Tg) or degree of cross-linking. In general, a copolymer with a lower Tg is less rigid and a copolymer with a higher Tg is more rigid. The tack of PSAs can be altered by the addition of components to alter the viscosity or mechanical properties. Exemplary pressure sensitive adhesives are described in Czech et al., “Pressure-Sensitive Adhesives for Medical Applications,” in Wide Spectra of Quality Control, Dr. Isin Akyar (Ed., published by InTech), Chapter 17 (2011), which is hereby incorporated by reference in its entirety.

In one exemplary technique, a photosensitizer is applied to the tissue (e.g., Rose Bengal (RB) at concentration of less than 1.0% weight per volume in a buffer, e.g., phosphate buffered saline to form a skin tissue-RB complex), and then the tissue is irradiated with electromagnetic energy to produce a seal (e.g., irradiated at a wavelength of at least 488, at less than 2000 J/cm<2>, and/or at less than 1.5 W/cm<2>, e.g., about 0.6 W/cm<2>). This exemplary technique is described in U.S. Pat. No. 7,073,510, which is incorporated by reference in its entirety. In another exemplary technique, a laser can be used for tissue welding. In yet another exemplary technique, a photochemical agent is applied to the tissue, and then the tissue is irradiated with visible light to produce a seal.

According to one embodiment of the present invention, therapeutic agents can be integrated within the stretching/compression device to be released to the skin's holes to accelerate healing thereof. Exemplary agents include one or more growth factors (e.g., vascular endothelial growth factor (VEGF), platelet-derived growth factor (PDGF), transforming growth factor beta (TGF-B), fibroblast growth factor (FGF), epidermal growth factor (EGF), and keratinocyte growth factor); one or more stem cells (e.g., adipose tissue-derived stem cells and/or bone marrow-derived mesenchymal stem cells); steroids (for example, steroids to prevent edema), agents which prevent post-inflammatory skin hyperpigmentation (e.g., hydroquinone, azelaic acid, kojic acid, mandelic acid, or niacinamide); one or more analgesics (e.g., paracetamol/acetaminophen, aspirin, a non-steroidal anti-inflammatory drug, as described herein, a cyclooxygenase-2-specific inhibitor, as described herein, dextropropoxyphene, co-codamol, an opioid (e.g., morphine, codeine, oxycodone, hydrocodone, dihydromorphine, pethidine, buprenorphine, tramadol, or methadone), fentanyl, procaine, lidocaine, tetracaine, dibucaine, benzocaine, p-butylaminobenzoic acid 2-(diethylamino) ethyl ester HCl, mepivacaine, piperocaine, dyclonine, or venlafaxine); one or more antibiotics (e.g., cephalosporin, bactitracin, polymyxin B sulfate, neomycin, bismuth tribromophenate, or polysporin); one or more antifungals (e.g., nystatin); one or more anti-inflammatory agents (e.g., a non-steroidal anti-inflammatory drug (NSAID, e.g., ibuprofen, ketoprofen, flurbiprofen, piroxicam, indomethacin, diclofenac, sulindac, naproxen, aspirin, ketorolac, or tacrolimus), a cyclooxygenase-2-specific inhibitor (COX-2 inhibitor, e.g., rofecoxib (Vioxx®), etoricoxib, and celecoxib (Celebrex®)), a glucocorticoid agent, a specific cytokine directed at T lymphocyte function), a steroid (e.g., a corticosteroid, such as a glucocorticoid (e.g., aldosterone, beclometasone, betamethasone, cortisone, deoxycorticosterone acetate, dexamethasone, fludrocortisone acetate, hydrocortisone, methylprednisolone, prednisone, prednisolone, or triamcinolone) or a mineralocorticoid agent (e.g., aldosterone, corticosterone, or deoxycorticosterone)), or an immune selective anti-inflammatory derivative (e.g., phenylalanine-glutamine-glycine (FEG) and its D-isomeric form (feG))); one or more antimicrobials (e.g., chlorhexidine gluconate, iodine (e.g., tincture of iodine, povidone-iodine, or Lugol's iodine), or silver, such as silver nitrate (e.g., as a 0.5% solution), silver sulfadiazine (e.g., as a cream), or Ag<+> in one or more useful carriers (e.g., an alginate, such as Acticoat® including nanocrystalline silver coating in high density polyethylene, available from Smith & Nephew, London, U.K., or Silvercel® including a mixture of alginate, carboxymethylcellulose, and silver coated nylon fibers, available from Systagenix, Gatwick, U.K.; a foam (e.g., Contreet® Foam including a soft hydrophilic polyurethane foam and silver, available from Coloplast A/S, Humlebaek, Denmark); a hydrocolloid (e.g., Aquacel® Ag including ionic silver and a hydrocolloid, available from Conva Tec Inc., Skillman, N.J.); or a hydrogel (e.g., Silvasorb® including ionic silver, available from Medline Industries Inc., Mansfield, Mass.)); one or more antiseptics (e.g., an alcohol, such as ethanol (e.g., 60-90%), 1-propanol (e.g., 60-70%), as well as mixtures of 2-propanol/isopropanol; boric acid; calcium hypochlorite; hydrogen peroxide; manuka honey and/or methylglyoxal; a phenol (carbolic acid) compound, e.g., sodium 3,5-dibromo-4-hydroxybenzene sulfonate, trichlorophenylmethyl iodosalicyl, or triclosan; a polyhexanide compound, e.g., polyhexamethylene biguanide (PHMB); a quaternary ammonium compound, such as benzalkonium chloride (BAC), benzethonium chloride (BZT), cetyl trimethylammonium bromide (CTMB), cetylpyridinium chloride (CPC), chlorhexidine (e.g., chlorhexidine gluconate), or octenidine (e.g., octenidine dihydrochloride); sodium bicarbonate; sodium chloride; sodium hypochlorite (e.g., optionally in combination with boric acid in Dakin's solution); or a triarylmethane dye (e.g., Brilliant Green)); one or more antiproliferative agents (e.g., sirolimus, tacrolimus, zotarolimus, biolimus, or paclitaxel); one or more emollients; one or more hemostatic agents (e.g., collagen, such as microfibrillar collagen, chitosan, calcium-loaded zeolite, cellulose, anhydrous aluminum sulfate, silver nitrate, potassium alum, titanium oxide, fibrinogen, epinephrine, calcium alginate, poly-N-acetyl glucosamine, thrombin, coagulation factor(s) (e.g., II, V, VII, VIII, IX, X, XI, XIII, or Von Willebrand factor, as well as activated forms thereof), a procoagulant (e.g., propyl gallate), an anti-fibrinolytic agent (e.g., epsilon aminocaproic acid or tranexamic acid), and the like); one or more procoagulative agents (e.g., any hemostatic agent described herein, desmopressin, coagulation factor(s) (e.g., II, V, VII, VIII, IX, X, XI, XIII, or Von Willebrand factor, as well as activated forms thereof), procoagulants (e.g., propyl gallate), antifibrinolytics (e.g., epsilon aminocaproic acid), and the like); one or more anticoagulative agents (e.g., heparin or derivatives thereof, such as low molecular weight heparin, fondaparinux, or idraparinux; an anti-platelet agent, such as aspirin, dipyridamole, ticlopidine, clopidogrel, or prasugrel; a factor Xa inhibitor, such as a direct factor Xa inhibitor, e.g., apixaban or rivaroxaban; a thrombin inhibitor, such as a direct thrombin inhibitor, e.g., argatroban, bivalirudin, dabigatran, hirudin, lepirudin, or ximelagatran; or a coumarin derivative or vitamin K antagonist, such as warfarin (coumadin), acenocoumarol, atromentin, phenindione, or phenprocoumon); one or more immune modulators, including corticosteroids and non-steroidal immune modulators (e.g., NSAIDS, such as any described herein); one or more proteins; or one or more vitamins (e.g., vitamin A, C, and/or E).

For the skin tightening methods described herein, the use of anticoagulative and/or procoagulative agents may be of particular relevance. For instance, by controlling the extent of bleeding and/or clotting in the incisions and/or excisions, the skin tightening effect can be more effectively controlled. Thus, in some embodiments, the methods and devices herein include one or more anticoagulative agents, one or more procoagulative agents, one or more hemostatic agents, or combinations thereof. In particular embodiments, the therapeutic agent controls the extent of bleeding and/or clotting in the treated skin region, including the use one or more anticoagulative agents (e.g., to inhibit clot formation prior to skin healing or slit/hole closure) and/or one or more hemostatic or procoagulative agents.

### Methods for Treating Skin Regions

The present invention relates to methods and devices that can be applied to treated skin regions. In particular embodiments, these regions are treated with one or more procedures to improve skin appearance. Accordingly, the stretching/compression device, and methods herein can be useful for skin rejuvenation (e.g., removal of pigment, tattoo removal, veins (e.g., spider veins or reticular veins), and/or vessels in the skin) or for treating acne, allodynia, blemishes, ectopic dermatitis, hyperpigmentation, hyperplasia (e.g., lentigo or keratosis), loss of translucency, loss of elasticity, melasma (e.g., epidermal, dermal, or mixed subtypes), photodamage, rashes (e.g., erythematous, macular, papular, and/or bullous conditions), psoriasis, rhytides (or wrinkles, e.g., crow's feet, age-related rhytides, sun-related rhytides, or heredity-related rhytides), sallow color, scar contracture (e.g., relaxation of scar tissue), scarring (e.g., due to acne, surgery, or other trauma), skin aging, skin contraction (e.g., excessive tension in the skin), skin irritation/sensitivity, skin laxity (e.g., loose or sagging skin or other skin irregularities), striae (or stretch marks), vascular lesions (e.g., angioma, erythema, hemangioma, papule, port wine stain, rosacea, reticular vein, or telangiectasia), or any other unwanted skin irregularities.

Such treatments can be included any parts of the body, including the face (e.g., eyelid, cheeks, chin, forehead, lips, or nose), neck, thighs, chest (e.g., as in a breast lift), arms, legs, nose, forehead, buttocks, and/or back. Accordingly, the devices on the invention can be arranged or configured to be amenable to the size or geometry of different body regions. Such arrangements and configurations can include any useful shape (e.g., linear, curved, or stellate), size, and/or depth.

In some embodiments, the incised or excised tissue portions forms a hole in the skin region, where the diameter or width of the hole is less than about 1.0 mm and results in a tissue portion having a diameter or width that is less than about 2.0 mm. In further embodiments, the tissue portion has a diameter or width that is less than about 2.0 mm and a length of more than about 1.0 mm. In particular embodiments, relatively small dimensions of the tissue portions can promote healing while minimizing the formation of scars.

Furthermore, the fractional treatment resulting in a plurality of tissue portions can be incised or excised in any beneficial pattern within the skin region. Exemplary patterns within the skin region include tile patterns or fractal-like shapes, where the array of hollow tubes can be arranged, e.g., in a base, to effectuate such a pattern (see FIGS. 7-9). It should be emphasized that according to one embodiment of the present invention, there can be an overlap between one coring step to the other (by vertex or facet); and, according to another embodiment of the present invention there can an overlap in the cross section between consecutive coring steps (as can be seen in FIG. 7). In other words, the first cross section area of the first coring step is, as shown, e.g., in FIG. 7, is hexagonal. The next step, according to one embodiment of the present invention, could provide coring in any location within said hexagonal cross section of the first step.

According to another embodiment of the present invention, a higher density and/or smaller spacing of tissue portions (e.g., slits and/or holes) can be incised or excised in the skin in the center of the pattern or in thicker portions of the skin. In another example, the pattern within the skin can be random, staggered rows, parallel rows, a circular pattern, a spiral pattern, a square or rectangular pattern, a triangular pattern, a hexagonal pattern, a radial distribution, or a combination of one or more such patterns of the incised or excised tissue portions. The pattern can arise from modifications to the average length, depth, or width of an incised or excised tissue portion, as well as the density, orientation, and spacing between such incisions and/or excisions (e.g., by using an apparatus having one or more blades or tubes with differing lengths, widths, or geometries that are arranged in a particular density or spacing pattern). Such patterns can be optimized to promote unidirectional, non-directional, or multidirectional contraction or expansion of skin (e.g., in the x-direction, y-direction, x-direction, x-y plane, y-z plane, x-z plane, and/or xyz-plane), such as by modifying the average length, depth, width, density, orientation, and/or spacing between incisions and/or excisions.

Any useful portion of the skin can be incised or excised. Such tissue portions can include epidermal tissue, dermal tissue, and/or cells or tissue proximal to the dermal/fatty layer boundary (e.g., stem cells).

According to another embodiment of the present invention, the holes in the tissue (resulting in removing tissue or one or more tissue portions from a skin region—the excised tissue) could be achieved by using a scalpel, application of energy (e.g., laser), coblation, coagulation, ultrasound, microwave energy, RF, application of heat (to evaporate skin portions), mechanical applicator that ‘drills’ through the skin whilst suction is applies (during the drilling or thereafter) to removes the excised skin portion, or any another instrument. For example, an excision includes any removed tissue or tissue portion from a skin region, which can result in excised tissue portions having a particular geometry (e.g., a cylindrical geometry, rectangular, triangle etc. or any arbitrary shape) and produce one or more holes (i.e., negative space created by the removal of tissue) in the skin region. Exemplary methods of forming excised tissue portions or excisions include use of one or more hollow needles (optionally include one or more notches, extensions, protrusions, and/or barbs), one or more microaugers, one or more microabraders, any useful tool for forming excisions, or any methods and apparatuses described herein.

### Safety Subsystem

According to one embodiment of the present invention, the following safety issues are taken into account.
Emergency Power Off switch that immediately removes all energy and motions from the system
all operative robotic arms stopes and descends slowly to rest in case of total power loss
Needles/Punches are automatically retracted to safe location within mechanism in case of loss of power
all Robotics arms are integrated with force sensors that can detect excessive forces and stop immediately
speed of movement is limited during treatment to below 500 mm/sec and below 50 mm/sec while moving from one coring location to another
movements during coring are limited to 20 mm and maximum allowed orientation is less than 10 degrees
Imaging system continuously monitors distance between punches and skin
All computer-controlled movements are initiated by the user. These movements can be quickly stopped via the user interface.

Reference is now made to FIG. 17 illustrating histological analysis-cross tissue sections after 0, 2 and 5 weeks post the fractional coring (tissue removal) treatment.

As can be seen in the figure, immediately after the treatment (at 0 weeks), fractional holes have been created post the excision of the cored tissue.

After 2 and 5 weeks, healing including fibroblasts migration and collagen synthesis as well as maturation occurred and the skin was tightened.

According to another embodiment of the present invention the excised tissue could be according to any embodiment as disclosed above, however, the directional tightening thereof could also be performed by application of at least one energy source being selected from a group consisting of application of temperature to heat and evacuate tissue, application of laser, RF, coblation, coagulation, microwave energy, ultrasound, application of any other type of energy and any combination thereof.

In such an embodiment, for example, an RF electrode could be applied either to the entire treated skin region or to the area between each excised region

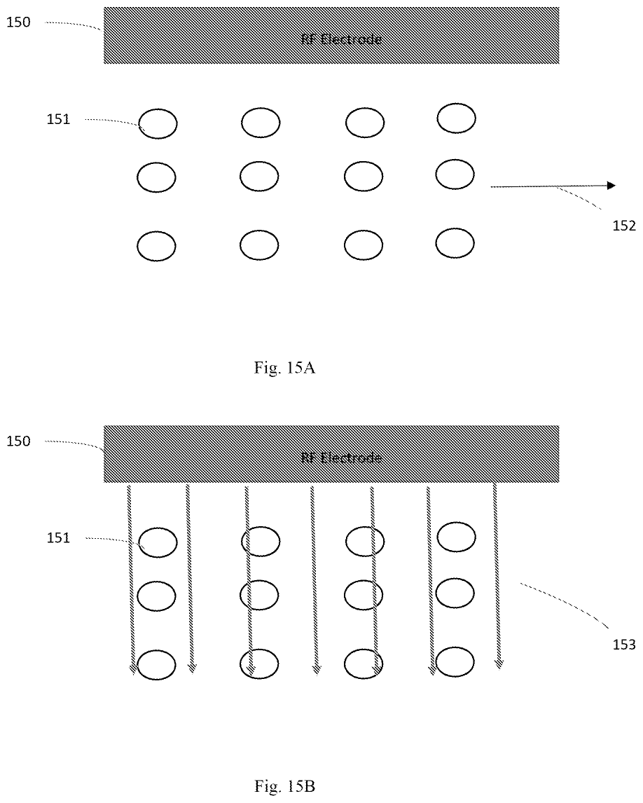

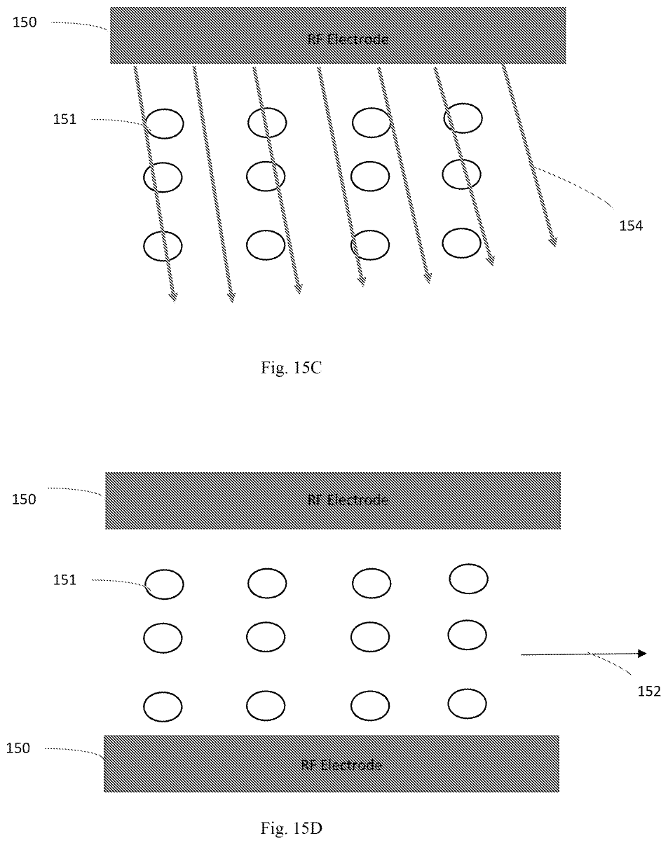

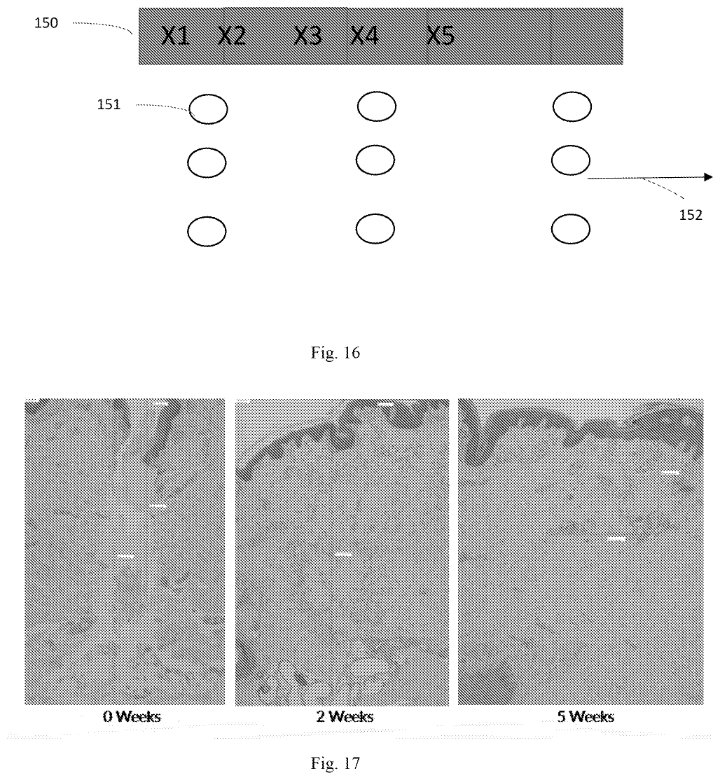

Reference is now made to FIGS. 15-16 schematically illustrating such an embodiment.

In FIG. 15a schematically illustrated the skin region in which plurality of excisions 150 have been produced. In this figure, also integrated is an RF electrode 150 which post the excision are adapted to apply energy to the skin to provide the directional tightening. It is within the scope of the present invention that once the RF energy is applied to the tissue a different magnetic field would be created in between the excised tissue so as to provide skin tightening (see arrow 152).

It should be noted that the energy applied by the RF electrode (or a different energy source) could be e.g., as illustrated in FIG. 15b (see arrow 153) or 15c (see arrow 154).

According to another embodiment, when applicable, 2 RF electrodes are employed (each from a different side of the skin), see FIG. 15d.

Reference is now made to FIG. 16 which schematically illustrates another embodiment of the present invention, in which the energy applied to the skin tissue (in this case RF energy) is divided into several segments (FIG. 16 illustrates 5 segments X1 . . . . X5), each section is adapted to apply a different amount of energy to the tissue. Such energy level could be adjusted to optimize the treatment.

It should be emphasized that although FIGS. 15-16 illustrates RF electrode and RF energy, the same applies to application of laser, RF, pulsed electromagnetic field, coblation, coagulation, microwave energy, ultrasound, application of any other type of energy and any combination thereof.

Combined Energy-Based Coring with Mechanical-Based Coring

According to another embodiment of the present invention, the punches/needles are also adapted to apply RF energy to the skin and tissue.

According to such an embodiment, the punches/needles are adapted to penetrate and core the skin (to produce a plurality of excised tissue portions) and either simultaneously or sequentially deliver RF energy to provide heat to the tissue and to fractional ablate/coagulate the tissue. In such an embodiment, the punches/needles are basically an RF electrode as well as a cutting element.

It is within the scope of the present invention, where the application of RF energy to the skin will facilitate the tissue excision as well as apply ablative and coagulative wound healing derived impact to the tissue.

According to one embodiment, each punch/needle is in communication with at least one RF generator.

According to another embodiment, all punches/needles are in communication with at least one RF generator.

According to another embodiment of the present invention, pulsed electromagnetic frequency generator is in communication with at least one of said punches/needles. According to another embodiment, the pulsed electromagnetic frequency generator is adapted to provide a dynamic magnetic field such that electromagnetic pulses are delivered to said region of a patient's skin.

According to another embodiment, said electromagnetic pulses vary with time.

According to another embodiment, the dynamic magnetic field is provided by means of at least one coil. According to another embodiment, at least one of the punches/needles is at least partially coiled by at least one coil. According to another embodiment, all the punches/needles are at least partially coiled by one coil.

According to another embodiment of the present invention, all of said punches/needles are adapted to simultaneously provide said electromagnetic pulses to said region of a patient's skin and apply RF energy. According to one embodiment of the present invention said RF energy is provided in the shape of heat to said region of a patient's skin.

According to another embodiment of the present invention, a control unit monitors and/or controls said the application of heat (by means of the RF energy) to the tissue within said region of skin.

According to another embodiment of the present invention, the shape of said electromagnetic pulse is selected from the group consisting of square wave, a sine wave, a triangular wave, sawtooth wave, ramp waves, spiked wave or any combination thereof.

According to another embodiment of the present invention, the magnetic field intensity B of each pulse applied by said pulsed electromagnetic frequency generator ranges between about 0 and about 3 Tesla.

According to another embodiment of the present invention, the magnetic field intensity B of each pulse applied by said pulsed electromagnetic frequency generator ranges between about 0 to 40 Gauss.

According to another embodiment of the present invention, the duration of each pulse applied by said pulsed electromagnetic frequency generator ranges between about 3 and about 1000 milliseconds.

According to another embodiment of the present invention, the frequency F applied by the pulses of said pulsed electromagnetic frequency generator ranges between about 1 Hz and about 40 MHz.

According to another embodiment of the present invention, the energy E applied by the pulses of said pulsed electromagnetic frequency generator ranges between about 1 and about 150 watts per pulse or any combination thereof.

According to another embodiment of the present invention, the frequency F applied by the pulses applied by said step of applying pulsed electromagnetic therapy to said region to be higher than about 1 and lower than about 1M Hz.

According to another embodiment of the present invention, the frequency F applied by said electromagnetic field pulses ranges between 1 Hz and 50 Hz.

According to another embodiment of the present invention, the frequency of said RF energy ranges between 200 kHz and 10 MHz.

According to another embodiment of the present invention, the power P applied by said RF energy pulses ranges between 1 W and 100 W of RMS average power.

According to another embodiment of the present invention, at least one temperature sensor is provided.

According to another embodiment of the present invention, the temperature T the tissue reaches is higher than about 30 and lower than about 100 degrees.

According to another embodiment of the present invention, a mechanism for skin cooling is provided to regulate the temperature of the skin (applied by the RF energy).

According to another embodiment of the present invention, the device additionally comprising at least one RF electrode (in addition to the coring element; namely, the punches/needles) adapted to apply RF energy to the skin and tissue.

According to such an embodiment, the punches/needles are adapted to penetrate and core the skin (to produce a plurality of excised tissue portions) while the RF electrode either simultaneously or sequentially deliver RF energy to provide heat to the tissue and to fractional ablate/coagulate the tissue.

It is within the scope of the present invention, where the application of RF energy to the skin will facilitate the tissue excision as well as apply ablative and coagulative therapy to the tissue. According to one embodiment, the RF electrode is in communication with at least one RF generator.

According to another embodiment of the present invention, pulsed electromagnetic frequency generator is in communication with the at least one RF electrode. According to another embodiment, the pulsed electromagnetic frequency generator is adapted to provide a dynamic magnetic field such that electromagnetic pulses are delivered to said region of a patient's skin. According to another embodiment, said electromagnetic pulses vary with time.

According to another embodiment, the dynamic magnetic field is provided by means of at least one coil. According to another embodiment, at least one of the RF electrodes is at least partially coiled by at least one coil. According to another embodiment, all the RF electrodes are at least partially coiled by one coil.

According to another embodiment of the present invention, all of said RF electrodes are adapted to simultaneously provide said electromagnetic pulses to said region of a patient's skin and apply Rf energy. According to one embodiment of the present invention said RF energy is provided in the shape of heat to said region of a patient's skin.

According to another embodiment of the present invention, a control unit monitors and/or controls said the application of heat (by means of the RF energy) to the tissue within said region of skin.

According to another embodiment of the present invention, the shape of said electromagnetic pulse is selected from the group consisting of square wave, a sine wave, a triangular wave, sawtooth wave, ramp waves, spiked wave or any combination thereof.

According to another embodiment of the present invention, the magnetic field intensity B of each pulse applied by said pulsed electromagnetic frequency generator ranges between about 0 and about 3 Tesla.

According to another embodiment of the present invention, the magnetic field intensity B of each pulse applied by said pulsed electromagnetic frequency generator ranges between about 0 to 40 Gauss.

According to another embodiment of the present invention, the duration of each pulse applied by said pulsed electromagnetic frequency generator ranges between about 3 and about 1000 milliseconds.

According to another embodiment of the present invention, the frequency F applied by the pulses of said pulsed electromagnetic frequency generator ranges between about 1 Hz and about 40 MHz.

According to another embodiment of the present invention, the energy E applied by the pulses of said pulsed electromagnetic frequency generator ranges between about 1 and about 150 watts per pulse or any combination thereof.

According to another embodiment of the present invention, the frequency F applied by the pulses applied by said step of applying pulsed electromagnetic therapy to said region to be higher than about 1 and lower than about 1M Hz.

According to another embodiment of the present invention, the frequency F applied by said electromagnetic field pulses ranges between 1 Hz and 50 Hz.

According to another embodiment of the present invention, the frequency of said RF energy ranges between 200 kHz and 10 MHz.

According to another embodiment of the present invention, the power P applied by said RF energy pulses ranges between 1 W and 100 W of RMS average power.

According to another embodiment of the present invention, at least one temperature sensor is provided.

According to another embodiment of the present invention, the temperature T the tissue reaches is higher than about 30 and lower than about 100 degrees.

According to another embodiment of the present invention, a mechanism for skin cooling is provided to regulate the temperature of the skin (applied by the RF energy).

### Impedance/Temperature Measurements

According to another embodiment of the present invention, at least one impedance/temperature sensor(s) is embedded in the distal-most end of at least one of the punches to provide indication as to the depth of penetration of each of at least one of the punches. Such information can be utilized to indicate if each punch is within the preferred treatment zone or outside thereof.

### Cutting Element

According to another embodiment of the present invention, the skin coring instrument (namely, the punches/needles) comprise at least one cutting element (e.g., at least one blade), adapted to grind/mil the cored/excised tissue so as to facilitate extraction thereof.

The at least one cutting element could be integrated in the punches/needles or in communication therewith.

As stated above, according to one object of the present invention, the system comprises at least one vacuum subsystem adapted to apply suction to remove excising portions of said skin tissue.

Combining the at least one cutting element in the system will facilitate the extraction of the excised tissue by said vacuum subsystem. Alternatively, the cutting element will facilitate the removal of the cored/excised tissue with the aid of the retention member.

### Injectable Matter

According to another embodiment of the present invention, at least one needle is provided with the punches, to inject treatment substances to the treatment area.

According to another embodiment of the present invention, the punches are needles adapted to inject treatment substances to the treatment area.

According to another embodiment of the present invention, the needles could be with either of a homogeneous/heterogeneous size.

According to another embodiment of the present invention, the substance could be selected from a group consisting of hyaluronic acid, botox, collagen, stem cells or any of the adhesives described above.

Thus, it is within the scope of the present invention to provide a method of directional skin tightening of a skin region, comprising:
(ii) producing a plurality of excised tissue portions in a region of skin tissue; and,
(ii) applying energy to said skin region to provide contraction or expansion of said skin region in a predetermined direction; thereby providing directional skin tightening in said skin tissue.

It is another object of the present invention to provide the method as defined above, additionally comprising step of applying stretching tension to said skin region before said step of producing a plurality of excised tissue portions.

It is another object of the present invention to provide the method as defined above, wherein said directional skin tightening is performed at a direction selected from a group consisting of x-, y-, and/or z-direction and any combination thereof.

It is another object of the present invention to provide the method as defined above, wherein said step of producing a plurality of excised tissue portions in a region of skin tissue is performed by means selected from a group consisting of mechanical means, application of temperature to heat and evacuate tissue, application of laser, RF, pulsed electromagnetic field, coblation, coagulation, microwave energy, ultrasound, application of any other type of energy and any combination thereof.

It is another object of the present invention to provide the method as defined above, wherein said step of applying energy to said skin region to provide contraction or expansion of said skin region is performed by means selected from a group consisting of mechanical means, application of temperature to heat and evacuate tissue, application of laser, RF, pulsed electromagnetic field, coblation, coagulation, microwave energy, ultrasound, application of any other type of energy and any combination thereof.

It is another object of the present invention to provide the method as defined above, wherein said step of producing a plurality of excised tissue portions in a region of skin tissue is performed by a system comprising at least one robotic arm, said at least one robotic arm comprising at least one skin coring instrument.

It is another object of the present invention to provide the method as defined above, wherein said at least one skin coring instrument comprising a plurality of punches configured to contact a surface of the skin to generate holes in the skin tissue by excising portions of the skin tissue.

It is another object of the present invention to provide the device or method as defined above, wherein said plurality of punches are at least 7 punches; 6 of which are disposed at a hexagonal shape around a seventh central punch.

It is another object of the present invention to provide the device or method as defined above, wherein said plurality of punches are at least 6 punches; 5 of which are disposed at a pentagonal shape around a sixth central punch.

It is another object of the present invention to provide the method as defined above, wherein at least a portion of said plurality of punches are disposable.

It is another object of the present invention to provide the method as defined above, wherein said plurality of punches are adapted to penetrate said skin either in a simultaneously or sequentially manner.

It is another object of the present invention to provide the method as defined above, wherein said plurality of punches are characterized by either a similar or substantially different cross section area.

It is another object of the present invention to provide the method as defined above, wherein said plurality of punches are adapted to penetrate said skin to a depth of 1 to 4 mm.

It is another object of the present invention to provide the method as defined above, wherein at least a portion of said plurality of punches are characterized by a radius of 0.15 mm-2.0 mm.

It is another object of the present invention to provide the method as defined above, wherein said cross section area is selected from a group consisting of circular, rectangular, triangular, hexagonal, oval, staggered rows, parallel rows, a spiral pattern, a square or rectangular pattern, a radial distribution and any combination thereof.

It is another object of the present invention to provide the method as defined above, wherein said system additionally comprising at least one controller adapted to control the positioning of said at least one robotic arm relatively to said skin area.

It is another object of the present invention to provide the method as defined above, wherein said controller comprising at least one engine adapted to control at least one parameter selected from a group consisting of the rotation, translation, angle of penetration of said at least one robotic arm relatively to said skin, depth of penetration, coverage rate, the diameter of at least one excised tissue multiplied by number of cores, different area of said skin to be treated and any combination thereof.

It is another object of the present invention to provide the method as defined above, wherein said parameters are adjusted manually by the operator or automatically by said controller.

It is another object of the present invention to provide the method as defined above, wherein said parameters are real time adjusted.

It is another object of the present invention to provide the method as defined above, wherein said rotation is at a speed in the range of 1000-7000 RPM.

It is another object of the present invention to provide the method as defined above, wherein said translation is at a speed in the range of 0-500 mm/sec.

It is another object of the present invention to provide the method as defined above, wherein said translation of said at least one robotic arm relatively to said skin changes as said at least one robotic arm gets closer to said skin.

It is another object of the present invention to provide the method as defined above, wherein said rotation of said at least one robotic arm changes as said at least one robotic arm gets closer to said skin and penetrates said skin.

It is another object of the present invention to provide the method as defined above, wherein each punch of said plurality of punches rotates individually in a predefined direction in a predetermined speed.

It is another object of the present invention to provide the method as defined above, wherein said plurality of punches rotate simultaneously.

It is another object of the present invention to provide the method as defined above, wherein each punch of said plurality of punches translates individually.

It is another object of the present invention to provide the method as defined above, wherein said plurality of punches translate simultaneously.

It is another object of the present invention to provide the method as defined above, wherein said controller comprising stopping mechanism adapted to limit the depth to which at least a portion of said plurality of punches penetrate said skin.

It is another object of the present invention to provide the method as defined above, wherein said angle of penetration is substantially perpendicular to said skin.

It is another object of the present invention to provide the method as defined above, wherein said controller is adapted to define at least one no-fly zone; said no-fly zone is defined as an area to which said system provides no treatment.

It is another object of the present invention to provide the method as defined above, wherein said system additionally provide the skin with additives.

It is another object of the present invention to provide the method as defined above, wherein said additives are selected from a group consisting of therapeutic agents, saline solution growth factors, platelet-derived growth factor (PDGF), transforming growth factor beta (TGF-B), fibroblast growth factor (FGF), epidermal growth factor (EGF), and keratinocyte growth factor); one or more stem cells; steroids, agents which prevent post-inflammatory skin hyperpigmentation, hydroquinone, azelaic acid, kojic acid, mandelic acid, or niacinamide; one or more analgesics; one or more antifungals; one or more anti-inflammatory agents, or a mineralocorticoid agent, an immune selective anti-inflammatory derivative; one or more antimicrobials; a foam; or a hydrogel, one or more antiseptics, one or more antiproliferative agents, one or more emollients; one or more hemostatic agents, a procoagulant, an anti-fibrinolytic agent, one or more procoagulative, one or more anticoagulative agents, one or more immune modulators, including corticosteroids and non-steroidal immune modulators, one or more proteins; or one or more vitamins and any combination thereof.

It is another object of the present invention to provide the method as defined above, wherein said system additionally comprising at least one imaging subsystem adapted to guide said at least one skin coring instrument.

It is another object of the present invention to provide the method as defined above, wherein said imaging subsystem comprises at least one selected from a group consisting at least one camera, under skin imaging such as ultrasound-based imaging, OCT and any combination thereof.

It is another object of the present invention to provide the method as defined above, wherein said system additionally comprising at least one vacuum subsystem adapted to apply suction to remove excising portions of said skin tissue.

It is another object of the present invention to provide the method as defined above, wherein said skin could be part of a treatment area selected from a group consisting of forehead, cheeks, jaw line, neck, thighs, upper arms, tummy, abdomen, face, eyelid, nose, forehead, chin, forehead, lips, nose, neck, chest, legs, back and any combination thereof.

It is another object of the present invention to provide the method as defined above, wherein said method is used for focal elimination of redundant dermal tissue for skin tightening, at least partially scar removal, skin rejuvenation, at least partially removal of pigment, at least partially tattoo removal, veins, acne, allodynia, blemishes, ectopic dermatitis, hyperpigmentation, hyperplasia, lentigo or keratosis, loss of translucency, loss of elasticity, melasma, photodamage, psoriasis, rhytides, wrinkles, sallow color, scar contracture, scarring, wrinkles, folds, acne scars, dyschromia, striae, surgical scars, cellulite, tattoos removal, cheek wrinkles, facial wrinkles, facial folds, skin aging, skin contraction, skin irritation/sensitivity, skin laxity, striae, vascular lesions, angioma, erythema, hemangioma, papule, port wine stain, rosacea, reticular vein, or telangiectasia, or any other unwanted skin irregularities and any combination thereof.

It is another object of the present invention to provide the method as defined above, wherein said system utilizes at least one selected from a group consisting of mechanical visualization, OCT, Ultrasound, machine learning algorithms, artificial intelligence, image processing and any combination thereof to efficiency select the preferred location of the tissue to be treated to enhance outcome of said treatment.

It is another object of the present invention to provide the method as defined above, wherein an areal fraction of excised tissue portions is in the range of about 5% to about 30% of the skin region.

It is another object of the present invention to provide the method as defined above, wherein an areal fraction of excised tissue portions is less than about 10% of the skin region.

It is another object of the present invention to provide the method as defined above, comprising pre-stretching the skin region before producing the plurality of excised tissue.

It is another object of the present invention to provide a system of directional skin tightening of a skin region, comprising:
(i) means for producing a plurality of excised tissue portions in a region of skin tissue; and,
(ii) means for applying at least one type of energy to said skin region to provide contraction or expansion of said skin region in a predetermined direction, so as to provide directional skin tightening in said skin tissue.

It is another object of the present invention to provide the system as defined above, additionally comprising means for applying stretching tension to said skin region before said step of producing a plurality of excised tissue portions.

It is another object of the present invention to provide the system as defined above, wherein said directional skin tightening is performed at a direction selected from a group consisting of x-, y-, and/or z-direction and any combination thereof.

It is another object of the present invention to provide the system as defined above, wherein said means of producing a plurality of excised tissue portions in a region of skin tissue comprising means selected from a group consisting of mechanical means, application of temperature to heat and evacuate tissue, application of laser, RF, coblation, coagulation, microwave energy, pulsed electromagnetic field, ultrasound, application of any other type of energy and any combination thereof.

It is another object of the present invention to provide the system as defined above, wherein said means of applying energy to said skin region to provide contraction or expansion of said skin region comprising means selected from a group consisting of mechanical means, application of temperature to heat and evacuate tissue, application of laser, RF, pulsed electromagnetic field, coblation, coagulation, microwave energy, ultrasound, application of any other type of energy and any combination thereof.

It is another object of the present invention to provide the system as defined above, wherein said means of producing a plurality of excised tissue portions in a region of skin tissue comprising a system comprising at least one robotic arm, said at least one robotic arm comprising at least one skin coring instrument.

It is another object of the present invention to provide the system as defined above, wherein said at least one skin coring instrument (e.g. a skin corer) comprising a plurality of punches configured to contact a surface of the skin to generate holes in the skin tissue by excising portions of the skin tissue.

It is another object of the present invention to provide the system as defined above, wherein said plurality of punches are at least 7 punches; 6 of which are disposed at a hexagonal shape around a seventh central punch.

It is another object of the present invention to provide the system as defined above, wherein said plurality of punches are at least 6 punches; 5 of which are disposed at a pentagonal shape around a sixth central punch.

It is another object of the present invention to provide the system as defined above, wherein at least a portion of said plurality of punches are disposable.

It is another object of the present invention to provide the system as defined above, wherein said plurality of punches are adapted to penetrate said skin either in a simultaneously or sequentially manner.

It is another object of the present invention to provide the system as defined above, wherein said plurality of punches are characterized by either a similar or substantially different cross section area.

It is another object of the present invention to provide the system as defined above, wherein said plurality of punches are adapted to penetrate said skin to a depth of 1 to 4 mm.

It is another object of the present invention to provide the system as defined above, wherein at least a portion of said plurality of punches are characterized by a radius of 0.15 mm-2.0 mm.

It is another object of the present invention to provide the system as defined above, wherein said cross section area is selected from a group consisting of circular, rectangular, triangular, hexagonal, oval, staggered rows, parallel rows, a spiral pattern, a square or rectangular pattern, a radial distribution and any combination thereof.

It is another object of the present invention to provide the system as defined above, wherein said system additionally comprising at least one controller adapted to control the positioning of said at least one robotic arm relatively to said skin area.

It is another object of the present invention to provide the system as defined above, wherein said controller comprising at least one engine adapted to control at least one parameter selected from a group consisting of the rotation, translation, angle of penetration of said at least one robotic arm relatively to said skin, depth of penetration, coverage rate, the diameter of at least one excised tissue multiplied by number of cores, different area of said skin to be treated and any combination thereof.

It is another object of the present invention to provide the system as defined above, wherein said parameters are adjusted manually by the operator or automatically by said controller.

It is another object of the present invention to provide the system as defined above, wherein said parameters are real time adjusted.

It is another object of the present invention to provide the system as defined above, wherein said rotation is at a speed in the range of 1000-7000 RPM.

It is another object of the present invention to provide the system as defined above, wherein said translation is at a speed in the range of 0-500 mm/sec.

It is another object of the present invention to provide the system as defined above, wherein said translation of said at least one robotic arm relatively to said skin changes as said at least one robotic arm gets closer to said skin.

It is another object of the present invention to provide the system as defined above, wherein said rotation of said at least one robotic arm changes as said at least one robotic arm gets closer to said skin and penetrates said skin.

It is another object of the present invention to provide the system as defined above, wherein each punch of said plurality of punches rotates individually in a predefined direction in a predetermined speed. In some embodiments, the rotor is configured to rotate the punches at the same speed.

It is another object of the present invention to provide the system as defined above, wherein said plurality of punches rotate simultaneously.

It is another object of the present invention to provide the system as defined above, wherein each punch of said plurality of punches translates individually.

It is another object of the present invention to provide the system as defined above, wherein said plurality of punches translate simultaneously.

It is another object of the present invention to provide the system as defined above, wherein said controller comprising stopping mechanism adapted to limit the depth to which at least a portion of said plurality of punches penetrate said skin.

It is another object of the present invention to provide the system as defined above, wherein said angle of penetration is substantially perpendicular to said skin.

It is another object of the present invention to provide the system as defined above, wherein said controller is adapted to define at least one no-fly zone; said no-fly zone is defined as an area to which said system provides no treatment.

In some embodiments, a controller, as may be utilized to carry out control of one or more components, includes a processor configured to communicate with a non-transitory computer readable medium. The non-transitory computer readable medium is configurable as a memory which is configured to store instructions thereon, which, when executed by the processor, causes the processor to carry out instructions.

It is another object of the present invention to provide the system as defined above, wherein said system additionally provide the skin with additives.

It is another object of the present invention to provide the system as defined above, wherein said additives are selected from a group consisting of therapeutic agents, saline solution growth factors, platelet-derived growth factor (PDGF), transforming growth factor beta (TGF-B), fibroblast growth factor (FGF), epidermal growth factor (EGF), and keratinocyte growth factor); one or more stem cells; steroids, agents which prevent post-inflammatory skin hyperpigmentation, hydroquinone, azelaic acid, kojic acid, mandelic acid, or niacinamide; one or more analgesics; one or more antifungals; one or more anti-inflammatory agents, or a mineralocorticoid agent, an immune selective anti-inflammatory derivative; one or more antimicrobials; a foam; or a hydrogel, one or more antiseptics, one or more antiproliferative agents, one or more emollients; one or more hemostatic agents, a procoagulant, an anti-fibrinolytic agent, one or more procoagulative, one or more anticoagulative agents, one or more immune modulators, including corticosteroids and non-steroidal immune modulators, one or more proteins; or one or more vitamins and any combination thereof.

It is another object of the present invention to provide the system as defined above, wherein said system additionally comprising at least one imaging subsystem adapted to guide said at least one skin coring instrument.

It is another object of the present invention to provide the system as defined above, wherein said imaging subsystem comprises at least one selected from a group consisting at least one camera, under skin imaging such as ultrasound-based imaging, OCT and any combination thereof.

It is another object of the present invention to provide the system as defined above, wherein said system additionally comprising at least one vacuum subsystem adapted to apply suction to remove excising portions of said skin tissue.

It is another object of the present invention to provide the system as defined above, wherein said skin could be part of a treatment area selected from a group consisting of forehead, cheeks, jaw line, neck, thighs, upper arms, tummy, abdomen, face, eyelid, nose, forehead, chin, forehead, lips, nose, chest, legs, back and any combination thereof.

It is another object of the present invention to provide the system as defined above, wherein said system is used for focal elimination of redundant dermal tissue for skin tightening, at least partially scar removal, skin rejuvenation, at least partially removal of pigment, at least partially tattoo removal, veins, acne, allodynia, blemishes, ectopic dermatitis, hyperpigmentation, hyperplasia, lentigo or keratosis, loss of translucency, loss of elasticity, melasma, photodamage, psoriasis, rhytides, wrinkles, sallow color, scar contracture, scarring, wrinkles, folds, acne scars, dyschromia, striae, surgical scars, cellulite, tattoos removal, cheek wrinkles, facial wrinkles, facial folds, skin aging, skin contraction, skin irritation/sensitivity, skin laxity, striae, vascular lesions, angioma, erythema, hemangioma, papule, port wine stain, rosacea, reticular vein, or telangiectasia, or any other unwanted skin irregularities and any combination thereof.

It is another object of the present invention to provide the system as defined above, wherein said system utilizes at least one selected from a group consisting of mechanical visualization, OCT, Ultrasound, machine learning algorithms, artificial intelligence, image processing and any combination thereof to efficiency select the preferred location of the tissue to be treated to enhance outcome of said treatment.

It is another object of the present invention to provide the system as defined above, wherein an areal fraction of excised tissue portions is in the range of about 5% to about 30% of the skin region.

It is another object of the present invention to provide the system as defined above, wherein an areal fraction of excised tissue portions is less than about 10% of the skin region.

It is another object of the present invention to provide the system as defined above, comprising pre-stretching the skin region before producing the plurality of excised tissue.

According to another embodiment of the present invention, the skin coring instrument (namely, the punches/needles) comprise at least one cutting element (e.g., at least one blade), adapted to grind/mil the cored/excised tissue so as to facilitate extraction thereof. As stated above, according to one object of the present invention, the system comprises at least one vacuum subsystem adapted to apply suction to remove excising portions of said skin tissue. Combining the at least one cutting element (cutter) in the system will facilitate the extraction of the excised tissue by said vacuum subsystem. Alternatively, the cutting element will facilitate the removal of the cored/excised tissue with the aid of the retention member.

According to another embodiment of the present invention, once the coring of the tissue is performed, heat is applied to the treated area of the skin. The heat could be provided by means of RF energy, laser, electrical means and any combination thereof.

According to another embodiment, the heating element is the same as the coring element. According to another embodiment, the heating element is provided by means of a fiber optic coupled to a laser source, adapted to provide energy to the treated tissue post and/or during the coring phase.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, pharmacology, or related fields are intended to be within the scope of the invention.

1-336. (canceled)

337. A system of directional skin tightening of a skin region, comprising:
(i) an excisor configured to produce a plurality of excised tissue portions in a region of skin tissue; and
(ii) an applicator configured to apply at least one type of energy to said skin region in at least one predetermined direction to provide contraction or expansion of said skin region in a predetermined direction, so as to provide directional skin tightening in said skin tissue.

338. The system of claim 337, additionally comprising a tensioner configured to apply stretching tension to said skin region before said step of producing a plurality of excised tissue portions.

339. The system of claim 337, wherein said directional skin tightening is performed at a direction selected from a group consisting of the x-, y-, and/or z-direction and any combination thereof.

340. The system of claim 337, wherein said excisor is selected from a group consisting of mechanical means, application of temperature to heat and evacuate tissue, application of laser, RF, pulsed electromagnetic field, coblation, coagulation, microwave energy, ultrasound, application of any other type of energy and any combination thereof.

341. The system of claim 337, wherein said applicator is selected from the group consisting of mechanical means, application of temperature to heat and evacuate tissue, application of laser, RF, pulsed electromagnetic field, coblation, coagulation, microwave energy, ultrasound, application of any other type of energy and any combination thereof.

342. The system of claim 337, wherein said applicator is configured to apply said at least one type of energy at a different intensity, power, power density to each region of said skin tissue.

343. The system of claim 337, wherein said excisor is configured to include at least one robotic arm, said at least one robotic arm comprising at least one skin coring instrument.

344. The system of claim 337, wherein said at least one skin coring instrument comprises at least one selected from a group consisting of at least one needle, at least one punch and any combination thereof; said at least one skin coring instrument configured to contact a surface of the skin to generate holes in the skin tissue by excising portions of the skin tissue.

345. The system of claim 337, wherein at least a portion of said plurality of punches are disposable.

346. The system of claim 337, wherein said plurality of punches are adapted to penetrate said skin either in a simultaneously or sequentially manner.

347. The system of claim 337, wherein at least one of the following is held true: (a) said plurality of punches are characterized by either a similar or substantially different cross section area;
said cross section area is selected from a group consisting of circular, rectangular, triangular, hexagonal, oval, staggered rows, parallel rows, a spiral pattern, a square or rectangular pattern, a radial distribution and any combination thereof; (b) said plurality of punches are adapted to penetrate said skin to a depth of 1 to 4 mm; and (c) at least a portion of said plurality of punches are characterized by a radius of 0.15 mm-2.0 mm.

348. The system of claim 337, wherein said system additionally comprises at least one controller adapted to control the positioning of said at least one robotic arm relatively to said skin area; further wherein said controller comprises at least one engine adapted to control at least one parameter selected from a group consisting of the rotation, translation, an angle of penetration of said at least one robotic arm relatively to said skin, a depth of penetration, a coverage rate, a diameter of at least one excised tissue multiplied by number of cores, a different area of skin to be treated, and any combination thereof, and further wherein said parameters are adjusted manually by an operator or automatically by said controller.

349. The system of claim 337, wherein at least one of the following is held true: (a) said rotation is at a speed in the range of 1000-7000 RPM; said translation is at a speed in the range of 0-500 mm/sec; (c) said translation of said at least one robotic arm relatively to said skin changes as said at least one robotic arm gets closer to said skin; and (d) said rotation of said at least one robotic arm changes as said at least one robotic arm gets closer to said skin and penetrates said skin; and any combination thereof.

350. The system of claim 337, wherein either (a) each punch of said plurality of punches rotates individually in a predefined direction in a predetermined speed; or (b) said plurality of punches rotate simultaneously.

351. The system of claim 337, wherein either (a) each punch of said plurality of punches translates individually; or (b) said plurality of punches translate simultaneously.

352. The system of claim 348, wherein said controller comprising stopping mechanism adapted to limit the depth to which at least a portion of said plurality of punches penetrate said skin.

353. The system of claim 348, wherein said angle of penetration is substantially perpendicular to said skin.

354. The system of claim 348, wherein said controller is adapted to define at least one no-fly zone; said no-fly zone is defined as an area to which said system provides no treatment.

355. The system of claim 337, wherein said system additionally provides the skin with additives; wherein said additives are selected from a group consisting of therapeutic agents, saline solution growth factors, platelet-derived growth factor (PDGF), transforming growth factor beta (TGF-B), fibroblast growth factor (FGF), epidermal growth factor (EGF), and keratinocyte growth factor; one or more stem cells; steroids, agents which prevent post-inflammatory skin hyperpigmentation, hydroquinone, azelaic acid, kojic acid, mandelic acid, or niacinamide; one or more analgesics; one or more antifungals; one or more anti-inflammatory agents, or a mineralocorticoid agent, an immune selective anti-inflammatory derivative; one or more antimicrobials; a foam; or a hydrogel, one or more antiseptics, one or more antiproliferative agents, one or more emollients; one or more hemostatic agents, a procoagulant, an anti-fibrinolytic agent, one or more procoagulative, one or more anticoagulative agents, one or more immune modulators, including corticosteroids and non-steroidal immune modulators, one or more proteins, or one or more vitamins and any combination thereof.

356. The system of claim 337, wherein said system additionally comprises at least one imaging subsystem adapted to guide said at least one skin coring instrument; wherein said imaging subsystem comprises at least one selected group from a group consisting of at least one camera, under skin imaging such as ultrasound-based imaging, OCT and any combination thereof.

357. The system of claim 337, wherein said system additionally comprising at least one subsystem selected from a group consisting of (a) a vacuum subsystem adapted to apply suction to remove excising portions of said skin tissue; (b) at least one retainer, in communication with at least one of said excisor, adapted to contain said excised tissue, to avoid the use of a vacuum; (c) any combination thereof.

358. The system of claim 337, wherein said skin could be part of a treatment area selected from a group consisting of forehead, cheeks, jaw line, neck, upper arms, abdomen, face, eyelid, nose, forehead, chin, forehead, lips, nose, neck, thighs, chest, legs, back and any combination thereof.

359. The system of claim 337, wherein said system is used for focal elimination of redundant dermal tissue for skin tightening, at least partially scar removal, skin rejuvenation, at least partially removal of pigment, at least partially tattoo removal, veins, acne, allodynia, blemishes, ectopic dermatitis, hyperpigmentation, hyperplasia, lentigo or keratosis, loss of translucency, loss of elasticity, melasma, photodamage, psoriasis, rhytides, wrinkles, sallow color, scar contracture, scarring, wrinkles, folds, acne scars, dyschromia, striae, surgical scars, cellulite, tattoos removal, cheek wrinkles, facial wrinkles, facial folds, skin aging, skin contraction, skin irritation/sensitivity, skin laxity, striae, vascular lesions, angioma, erythema, hemangioma, papule, port wine stain, rosacea, reticular vein, or telangiectasia, or any other unwanted skin irregularities and any combination thereof.

360. The system of claim 337, wherein said system utilizes at least one selected from a group consisting of mechanical visualization, OCT, ultrasound, machine learning algorithms, artificial intelligence, image processing and any combination thereof to efficiently select the preferred location of the tissue to be treated to enhance the outcome of said treatment.

361. The system of claim 337, comprising pre-stretching the skin region before producing the plurality of excised tissue.

362. The system of claim 337, additionally comprising at least one cutter, integrated within said skin coring instrument, adapted to grind said excised tissue so as to facilitate extraction thereof.

363. An apparatus of fractional coring for directional skin tightening, comprising:
(i) an excisor a plurality of excised tissue portions in a region of skin tissue; and
(ii) a fastener configured to secure at least one portion of a stretching/compression device, having at least two portions, to the skin region adapted to provide contraction or expansion of said skin region in at least one predetermined direction; thereby endorsing collagen growth and providing directional skin tightening in said region of skin tissue.

364. The apparatus of claim 363, wherein the excisor comprises a micro-coring punch that is attached to a computer-controlled robotic arm capable of moving in six or more axes so as to provide at least six degrees of freedom.

365. The apparatus of claim 363, further comprising a video camera configured to provide visual feedback of at least the micro-coring punch and the skin and a closed-loop force sensor to determine when the punches break the skin.

366. The apparatus of claim 363, additionally comprising a cooler adapted to regulate the temperature of the skin.

367. The apparatus of claim 363, wherein the distal end of said at least one skin coring instrument additionally comprises at least one of at least one impedance sensor or at least one temperature sensor, wherein the at least one of the at least one impedance sensor or the at least one temperature sensor is adapted to provide an indication as to the depth of penetration of each of said at least one skin coring instrument.
